(19) United States
(12) Patent Application Publication
(10) Pub. No.: US 2024/0181139 A1
(43) Pub. Date: Jun. 6, 2024

(54) CHEMICAL TISSUE ABLATION WITH HYDROGEL MATRIX AND CORRESPONDING MEDICAL APPLICATIONS

(71) Applicant: Pramand LLC, Bedford, MA (US)

(72) Inventors: Payton Andrews, Marblehead, MA (US); Michael Bassett, Hampton, NH (US)

(21) Appl. No.: 18/226,169

(22) Filed: Jul. 25, 2023

Related U.S. Application Data
(60) Provisional application No. 63/430,577, filed on Dec. 6, 2022.

(51) Int. Cl. A61L 31/14; A61K 31/045; A61L 31/04; A61L 31/16

(52) U.S. Cl. CPC A61L 31/145; A61K 31/045; A61L 31/041; A61L 31/16; A61L 2300/20; A61L 2300/442

(57) ABSTRACT

Compositions, methods, and applications for ablative-chemical infused hydrogels are described. The hydrogels are biodegradable and can be formed in situ. The hydrogels are effective for delivering tissue ablative chemicals to target sites. The hydrogel delivered tissue ablative chemicals can be particularly effective for performing intrauterine ablation, even when fibroids are present, or for scarring fallopian tubes for birth control via minimally invasive procedures. The hydrogels can also be particularly effective at avoiding contact of tissue ablative chemicals with off-target tissues. The hydrogels can be installed using various delivery vehicles, including injections and catheters. The hydrogels can be designed to be retained in the target site for a period of hours to days, so that targeted tissue ablation can proceed in an ambulatory patient. Methods for transcervical installation of in situ formed hydrogels into the uterus or one or more fallopian tubes are described.

### CROSS REFERENCE TO RELATED APPLICATION

This application claims priority to copending U.S. provisional patent application 63/430,577 filed Dec. 6, 2022 to Andrews et al., entitled “Chemical Ablation With Hydrogel Matrix,” incorporated herein by reference.

### FIELD OF THE INVENTION

The invention relates to biocompatible hydrogels formed with a cytotoxic chemical in the hydrogel matrix for performing chemical ablation of tissue for medical treatment purposes. Generally, the hydrogels are biodegradable and can be formed in situ. The hydrogel delivered chemical ablation agents can be particularly effective for performing intrauterine ablation or for scarring fallopian tubes for birth control.

### BACKGROUND OF THE INVENTION

Abnormal uterine bleeding has been an identified medical problem since at least the second century AD. Early crude herbal type remedies have in recent times given way to ablation techniques using various energy sources to ablate the endometrial lining of the uterus. So called second generation techniques generally involved focused energy sources, such as lasers or radiofrequency thermal probes. The principle of ablating the uterine lining to stop abnormal bleeding was to reduce the need for hysterectomy to address the problem.

Third generation endometrial ablation pivoted to approaches attempting to address a comprehensive procedure which did not involve a health care professional targeting specific sites, a so called “global” approach. Then, the whole uterine lining could be treated at once, which was a benefit. Thermal balloons (e.g., THERMACHOICE®, Gynecare, Inc.) were marketed to treat the whole uterine lining at once using thermal ablation. Radiofrequency ablation and microwave energy based techniques were developed to treat the uterine lining more comprehensively. However these approaches use a pre-formed balloon or electrode array, or antenna, which may not completely conform to the uterine shape. Additionally, the presence of intrauterine polyps or fibroids also limits the applicability of such approaches. Some additional approaches attempt to overcome these limitations by instilling thermal solutions under controlled pressure. Using hot water or heated water vapor can be effective but presents safety hazards of burns to the cervix, vagina, and patient's thighs if improperly sealed. Cryo-endometrial ablation uses cooling to ablate the tissue and commercial systems have been developed based on this approach, but they again use a balloon based approach which may be limiting. Several alternative commercial products are competing using different technologies. Most of these approaches also use additional capital equipment, need significant patient anesthesia, and need to be done in a hospital setting, thus are limiting in these ways. These competing approaches are described in Wortmann, “Endometrial Ablation: Part, Present, and Future, Part II,” Surgical Technology International, 33:161-177 (November 2018).

In other contexts, ablation of tissue is seen as an alternative to more invasive surgery, which may be impractical or contraindicated, since ablative energy or solutions can be delivered through needles in a less invasive setting. Thus, ablations of tumors and other pathological lesions is routinely done using similar destructive energy and agents as mentioned above in the context of endometrial ablation. However, in addition, chemical ablation using agents that are toxic to tissue, such as trichloroacetic acid and alcohols, has been used in ablation or destruction of genital warts, precancerous tissue and various tumors. Concentrated alcohols are toxic, which is the basis for their use for ablation, so the use of chemical agents for ablation introduces a risk of harm to healthy tissue. Alcohols and other low viscosity aqueous solutions of tissue destructive agents are hard to localize after injection and tend to flow in any channels of low resistance. This can lead to undesirable side effects.

### BRIEF DESCRIPTION OF DRAWINGS

FIG. 1A is a depiction of a transcervical access system with dual syringes connected to a catheter via a Y-connector. The syringes in FIG. 1A are depicted as being asymmetric in diameter.

FIG. 1B is a fragmentary view of an optional T-branch connector of the transcervical access system of FIG. 1A.

FIG. 2A is a fragmentary view of a basic installation tip of the transcervical access system of FIG. 1.

FIG. 2B is a fragmentary view of an end-tapered installation tip of the transcervical access system of FIG. 1.

FIG. 2C is a fragmentary view of a tapered installation tip of the transcervical access system of FIG. 1.

FIG. 2D is a fragmentary view of a step-down installation tip of the transcervical access system of FIG. 1.

FIG. 3 is a depiction of a transcervical access system being used to deliver flowable components to the uterus through the cervix, with the optional cap element being used to control catheter placement and egress of material from the external cervical os.

FIG. 4A is a depiction of a separated configuration of a catheter and an egress limiter.

FIG. 4B is an illustration of a catheter assembly with a cervical plug and a connector.

FIG. 5 is a depiction of a catheter assembly with a luer connector and an egress limiter having an inflatable/deflatable adjustable cap element connected via an internal balloon lumen to an inflation/deflation port. In some embodiments, the inflatable/deflatable adjustable cap element may be positioned beyond the internal os of the cervix to limit egress into the cervix.

FIG. 6 is an illustration of a catheter assembly with a cervical plug, an egress limiter, and a connector.

FIG. 7 is a depiction of a transcervical procedure using a transcervical access system with a cervical plug and an egress limiter.

FIG. 8 is a depiction of a transcervical procedure using a transcervical access system with a cervical plug having a tapered tether or grip end.

FIG. 9A is a side view of a delivery system for injecting two in situ crosslinkable components to provide an alcohol-loaded hydrogel in a body lumen.

FIG. 9B is a cross-section of FIG. 9A along view line A-A.

FIG. 10 illustrates a method of using the apparatus of FIG. 9A to occlude fallopian tubes.

FIG. 11 is a plot of gel time as a function of pot life for a series of activated formulations having different volume percent alcohol.

FIG. 12 is a plot of the overall change in gel time as a function of percent ethanol for each activated formulation shown in FIG. 11.

FIG. 13 is a plot of the modulus as a function of time in accelerated days of gel samples there were prepared from an 8A15KPEG SG/8A20KPEG amine. HCl activated formulation having 72 vol % ethanol.

FIG. 14 is a plot of the modulus as a function of time in (real-time) days of gel samples there were prepared from an 8A15KPEG SS/8A20KPEG amine·HCl activated formulation having 72 vol % ethanol.

FIG. 15 is a plot of the modulus as a function of time in accelerated days of gel samples there were prepared from an 4A20KPEG SG/8A20KPEG amine·HCl activated formulation having 72 vol % ethanol.

FIG. 16 is a photograph of an ex-vivo pig cervix taken four hours after filling with a hydrogel that was 82.25 vol % ethanol.

FIG. 17 is a photograph of the pig cervix of FIG. 16 after sectioning along the sagittal plane.

FIG. 18 is a photograph of a side wall of the pig cervix of FIG. 17.

FIG. 19 is a magnified view of the side wall of FIG. 18.

FIG. 20 is an image of a section of a rabbit uterine horn after installation of a 72 wt % alcohol hydrogel.

FIG. 21. is an image of a section of a rabbit uterine horn after installation of a 72 wt % alcohol solution.

FIG. 22 is a photograph of one half of a dissected and TTC stained human uterus at 4 hours after installation of a 73.4 wt % alcohol hydrogel.

FIG. 23 is a close-up of the region in the dashed box of FIG. 22 that shows conformal fill of the alcohol hydrogel around a fibroid.

FIG. 24 is a photograph of a surgeon pulling on hydrogel adhered to ablated uterine tissue.

### SUMMARY OF THE INVENTION

In a first aspect, the invention pertains to a hydrogel comprising an insoluble crosslinked hydrophilic polymer with linear hydrophilic polymer arms and biodegradable linkages between one or more of the hydrophilic polymer arms. The hydrogel is imbibed/infused with a fluid comprising a tissue ablation composition at a concentration suitable to ablate tissue in contact with the hydrogel during a time period prior to disintegration of the hydrogel.

In a further aspect, the invention relates to a hydrogel-based system for chemical ablation. The system comprises a hydrogel precursor solution comprising a polyfunctional hydrophilic precursor macromolecular composition in a solution comprising a tissue ablation composition and an activator solution. Mixing the hydrogel precursor solution and the activator solution results in the crosslinking of the polyfunctional hydrophilic polymer to form a fluid imbibed/infused hydrogel. The fluid comprises the tissue ablation composition at a concentration suitable to ablate tissue in contact with the fluid imbibed/infused hydrogel.

In another aspect, the invention relates to a method comprising:
combining engorged microparticles with a solution of dissolved hydrogel precursors to form a hydrogel precursor solution, wherein the engorged particles are formed by absorbing a tissue ablation composition into polymer microparticles and
assembling an applicator with a reservoir of hydrogel precursor solution and a reservoir of activator solution configured for delivery of a mixture of hydrogel precursor solution and activator solution into a patient.

In additional aspects, the invention relates to a method comprising:
delivering a hydrogel formation solution comprising a dissolved blend of a polyfunctional hydrophilic hydrogel precursor, an activator, a solvent and a tissue ablation composition into a target localized domain in a patient, wherein a hydrogel forms in contact with a target tissue for ablation by the tissue ablation composition.

In additional aspects, the invention relates to a kit for forming tissue ablative hydrogels. The kit comprises a powder of a dry polyfunctional hydrophilic polymer with a plurality of crosslinkable functional groups, a liquid comprising a tissue ablation composition, wherein the powder is soluble in the liquid, and a container comprising an activator. Upon dissolving the powder in the liquid to form a hydrogel precursor solution and mixing the hydrogel precursor solution with the activator, a hydrogel formation solution is formed that gels into a biodegradable hydrogel comprising absorbed tissue ablation composition.

In further aspects, the invention relates to a method for forming a hydrogel precursor system for delivery of a tissue ablating hydrogel. The method comprises:
mixing a) a powder of a dry polyfunctional hydrophilic polymer with a plurality of crosslinkable functional groups and b) a liquid comprising a tissue ablation composition to dissolve the powder and form a hydrogel precursor solution and
providing the hydrogel precursor solution and an activator solution in a suitable applicator designed to mix the hydrogel precursor solution and the activator solution while delivering a blend of the hydrogel precursor solution and the activator solution to a target location within a patient.

In further aspects, the invention relates to a method for performing ablation of a uterine lining of a patient. The method comprises delivering a hydrogel formation solution through a catheter into a uterine cavity of the patient with an appropriate volume to fill the cavity without significant flow into the fallopian tubes and with restrictions to inhibit flow into the cervix. The hydrogel formation solution gels in no more than 20 seconds into a hydrogel engorged with tissue ablation composition at a concentration sufficient to ablate tissue in contact with the hydrogel.

### DETAILED DESCRIPTION OF THE INVENTION

Hydrogels have been identified as a vehicle for controlled delivery of ablative chemicals to provide for controlled, chemical-based ablation, such as for ablating the uterine lining with hydrogel matrix. Based on the deficiencies of existing approaches discussed above, an approach to endometrial ablation is desired that can be global, does not require significant patient anesthesia, can be performed in a lower intensity care setting with high safety and efficacy outcomes. In situ formed hydrogels have an advantage of conforming to the space, such as the uterine cavity, naturally through delivery as a flowable liquid and subsequent rapid crosslinking to fix the hydrogel into the conformal shape. The conformal nature of the hydrogels allows chemical ablation not to be contraindicated by tissue abnormalities in the uterus, such as fibroids. Thus, fibroids can be specifically targeted for ablation or ablated along with the uterus during uterine ablation. Hydrogels can be formulated such that they contain an ablative agent, such as trichloroacetic acid, acetic acid or alcohol, among others, without compromising its properties of in situ formation. To provide further options for the delivery of an ablating composition, the composition can be loaded into separate microparticles that are then loaded into the hydrogel. This microparticle delivery adapts some materials used for drug delivery for the delivery of the ablating chemicals. For ablating the uterine lining, the hydrogels can be delivered to be slightly expansive (increasing in volume) to ensure good coverage of the uterine cavity and contact with the uterine wall, while rapid crosslinking can prevent undesirable spreading of the ablative chemicals. Other gynecological applications for chemical ablation include scarring of fallopian tubes for birth control purposes, ablation of pre-cancerous cervical lesions, ablation of genital warts, as well as other locally present pathological tissues. The advantage of ablating these tissues “in situ” over surgical excision in several cases is the avoidance of associated weakening or modification of the organ which is being treated. For example, surgical excision of the cervix via a loop electrical excision procedure (LEEP) may result in a shortened cervix which can pre-dispose the patient to pre-term delivery.

While the hydrogel can be biodegradable and thus not requiring removal, the biodegradation timing and appropriate elution times allows for a relatively long time period for the chemical ablation effect. Since the hydrogels are usually resorbed by the patient, specialized tools are not needed for the removal of the hydrogel after completion of the ablation. Other ablative methods require the patient to be tethered to the energy or ablative agent source and must remain in the hospital bed for the duration of the treatment, while delivery of hydrogels containing an ablative agent can relieve this scheduling limitation and allow the patient to be ambulated, while the ablative effect takes place over hours. To effectuate the ablation, the hydrogel can be loaded with an ablative agent such as a cytotoxic drug or other agent, such as an alcohol. Additionally, agents such as trichloroacetic acid or acetic acid are also known to have contact toxicity with tissue. Hydrogels can be designed to have free radical crosslinking reactions with the acidic compounds, or acids can be loaded into microparticles for delivery with the hydrogels. Alcohols, such as ethanol, methanol, tert-butanol, and/or isopropyl alcohol, can be desirable since ethanol is a known metabolite with an extensive safety record of use and ingestion by humans. While the alcohol may gradually elute, the alcohol can remain localized within the hydrogel for a reasonable period of time based on entrapment in the hydrogel matrix with appropriate crosslink density and general hydrogel design. Due to the persistence of the hydrogel loaded with alcohol, the ablating alcohol is able to act over a longer period of time. Since, the alcohol is not immediately toxic at short times, such a procedure can be performed with reasonable levels of risk. Any off-target spillage can thus easily be controlled by removal of the alcohol infused hydrogel, which is entrapped in a solid configuration. The persistence of the hydrogel can also provide a physical barrier against the formation of adhesions between treated tissues and/or between treated tissues and off-target tissues. The concentration of alcohol can be adjusted somewhat to control the ablation effect, and it may be appropriate to make adjustments depending on the time in the patient's menstrual cycle, which may influence the target ablation thickness for intrauterine ablation and the dilation, position, and relative firmness of the cervix. Based on this control of cellular toxicity, this reduces risk from short contact with the loaded hydrogel at other tissue locations as well as mitigating harsh immediate effects experienced by the patient. The loaded hydrogels can then provide an effective alternative to other ablation technologies.

Alcohol can be metabolized by alcohol dehydrogenase to ameliorate its toxicity. Nevertheless, concentrated alcohol is an effective cytotoxin, which may be due to dehydration of cells and disrupting cellular functions. As such, alcohol has been used for ablating tissue through localized delivery. In general, its efficacy has been limited due to migration and dilution so that its effects can be less than desired since healthy non-target tissue can be destroyed or damaged unintentionally. The loaded hydrogels are designed to avoid this difficulty by localizing the hydrogel to reduce collateral damage while increasing efficacy for ablating the target tissue by maintaining the hydrogel across the entire surface of the target tissue for a selected range of time. Loaded hydrogels also allow for control of off-target ablation, allowing the user to easily remove any excess alcohol loaded hydrogel from unwanted site application. Removal of off-target alcohol loaded hydrogel may be performed, for example, by the use of suctioning, scraping, and/or flushing. In some embodiments, off-target sites can be coated with a barrier/release film prior to installation of the alcohol loaded hydrogel. Off-target sites may include the cervix and/or the fallopian tubes, for example. Based on extensive experience with medical hydrogels, the properties can be reasonably adjusted to provide the intended medical effects.

The hydrogels can be delivered inside the uterine cavity through an un-dilated or slightly dilated cervix using a small catheter, although the procedures herein can be similarly applied for a patient with a dilated cervix. The catheter can be a few millimeters in diameter, as explained further below. Contrary to some existing commercial ablation technologies which generally involve at least 6 mm dilation, dilation is either not needed or very minimal, and significant dilation may not be helpful since it is desirable to maintain containment of the hydrogel. Cervical dilation can prolong the ablation procedure and is painful if done rapidly, so not requiring dilation is a significant advantage. If the cervix is dilated prior to initiation of the procedure due to the patient's condition, appropriate adaptations can be made. The hydrogels can be delivered by combining a precursor solution comprising the alcohol and/or other ablation chemical(s), and an accelerator/activator solution used to initiate the crosslinking reactions. In some embodiments, the ablation composition can be split between the precursor solution and the activator solution, or delivered just with the activator solution. For delivery to fallopian tubes, catheters can be adapted for the delivery of the alcohol loaded hydrogels. For other applications, appropriate needles or catheters can be attached to the syringes or other delivery containers. At least one of the solutions can have a color, which can be provided by a dye, so that the hydrogel has improved visualization. In some embodiments, the dye is provided in the precursor solution to facilitate tracking of the alcohol component. The solutions can be dispensed from syringes and mixed immediately prior to delivery through a catheter attached to the syringes. The catheter system can be adapted to provide for optional suctioning and/or flushing to be performed prior to hydrogel delivery. In some embodiments, the adapted catheter system can provide for optional flushing or suctioning of off-target fluids and/or hydrogel. Generally, the procedure is suitable for performance in a doctor office to avoid the need for an operating room or other specialized procedure facility.

Hydrogels have been previously described for intrauterine delivery in the context of avoidance of adhesion formation. These are described in published U.S. patent application 2022/0143276 to Bassett et al. (hereinafter the '276 application), entitled “Hydrogels Formed In Situ and Composition Design for Intrauterine Use,” incorporated herein by reference. Transcervical access systems are described for hydrogel delivery in published U.S. patent application 2022/0142653 to Bassett et al., entitled “Transcervical Access Systems for Intrauterine Fluid Exchange, Such as Placement of Hydrogels In Situ,” which is attached as an Appendix.

With an appropriately infused hydrogel bolus, the hydrogel can conformally interface the uterine lining to provide longer term comprehensive contact for delivery of alcohol, or other ablative chemical, to the endometrial cells lining the uterus. In energy-based ablation procedures, the ablation is targeted to provide the ablation in relatively shorter periods of time associated with the surgical procedure. This allows for careful monitoring of the procedure, but it has the disadvantage that the ablation modality has an appropriate strength to affect the ablation in an appropriately focused time frame. In contrast, extending the ablation time beyond a surgical time frame provides for use of a weaker ablation modality to achieve an effective ablation outcome, which can reduce the risk of collateral damage since transient contact of the ablative chemical with other tissues may not be as likely to cause serious injury to the tissue.

Use of chemical ablation has been described for tumors based on absolute alcohol, and the side effects can be reduced by delivery of the treatment over several discrete sessions. Generally, the alcohol is simply injected into the location. See Saldanha et al., “Current Tumor Ablation Technologies: Basic Science and Device Review,” Seminars in Interventional Radiology, Vol. 27(3), 247-254 (2010), incorporated herein by reference. The Saldanha article also notes that acetic acid has been used for tumor ablation. For ethanol-based ablation of cervical dysplasia, cellulose has been described to help retain the ethanol at the site of delivery. See Mueller et al., “Optimizing Ethyl Cellulose-Ethanol Delivery Towards Enabling Ablation of Cervical Dysplasia,” Nature Scientific Reports, (2021) 11:16869, incorporated herein by reference. Acetic acid injected percutaneously has been found to be comparably effective as RF ablation for the treatment of small hepatocellular carcinoma. See Paul et al., “Acetic acid versus radiofrequency ablation for the treatment of hepatocellular carcinoma: a randomized controlled trial,” Diagnostic and Interventional Imaging, Vol. 101(2), 101-110, February 2020, incorporated herein by reference. The hydrogel systems described herein provide a convenient and controllable process for delivery of ablating alcohol or other ablating liquid. For injection into a tumor, a needle can be attached to the catheter applicator.

The hydrogels generally are designed to be biodegradable over a reasonable timeframe. If microparticles are used to facilitate delivery of the ablation composition, the microparticles can also be appropriately biodegradable. Since significant concentrations of alcohol (or other ablation compositions) are present, the hydrogels should be compatible with the alcohol with respect to solubility of precursors, ability to crosslink the precursors in the presence of the alcohol, and reasonable stability to degradation in the presence of the alcohols. In addition, the hydrogels should maintain the alcohols within the hydrogel matrix and elute the alcohol relatively slowly to allow for suitable ablation effect. In this way, hydrogel precursors can be delivered as liquids with rapid gelling to provide filling of the uterine cavity or other space, and the alcohol maintained in the hydrogel matrix can be held against and/or adhered to the uterine lining or other tissue to accomplish the desired ablation. The gradual elution of the alcohol from the hydrogel provides a natural termination of the ablation process, and the eluting alcohol should be diluted by physiological fluids upon migration from the hydrogel such that it should not present a significant risk to adjoining tissues. As the alcohol elutes out of the hydrogel, the water content of the hydrogel will increase to replace the eluted alcohol. The biodegradation of the hydrogel can proceed on a somewhat longer timescale to allow the tissue to heal and to reduce the risk of forming any adhesions, when administered in an intrauterine setting. Alcohols are exemplified, but other ablation compositions can be appropriately delivered. Established techniques for drug delivery are explained in the context of their adaptation for the delivery of ablative compositions.

A hydrogel is a material comprised of an insoluble hydrophilic polymer that when fully hydrated, is substantially comprised of water in a matrix of the insoluble hydrophilic polymer. Since the hydrogels formed in the procedures herein are hydrated when formed, further swelling with water may or may not occur, and significant swelling with water may not be desirable, as described further below. Medical hydrogels have been used for drug delivery, sealing, wound healing, space filing and other uses. Hydrogels may comprise materials that are absorbable/degradable or non-absorbable in contact with a patient's bodily fluids, and some hydrogels gradually hydrolyze to break down when in contact with water or other aqueous solution. The hydrogel is generally formed through the crosslinking/polymerization of soluble precursors, which may or may not be polymeric themselves. Some hydrogel systems allow for control of the crosslinking process through delay of crosslinking until hydrogel delivery, e.g., to ensure a large viscosity increase of materials flowing through a delivery device does not occur until after the hydrogel precursor mass is in place. These systems can involve the use of an external activation source that serves a dual purpose. First, it allows injectability of the hydrogel system with activation only after exiting the application device to prevent clogging of the catheter. Second, this allows working time prior to the injection event. In these embodiments, activation generally has been through the use of external radiation such as light to induce radical crosslinking or other energy input for thermally activated polymers, such as thermoreversible polymers or thermally induced radical polymerization. Thermally induced or UV induced crosslinking can be desirable for delivery of ablation chemicals, such as acetic acid or trichloroacetic acid, which may not be compatible with pH gated nucleophilic-electrophilic reactions. Hydrogel systems with external activation sources can be unwieldy, limiting certain clinical usage where one-handed application or fine dexterity is required, as well as taking longer procedural times. Additionally thermoreversible polymers, which use a single component, do not form hydrogels with mechanical integrity and modulus that is suitable for many medical device and longer persisting drug delivery applications. Free radical polymerization to drive the crosslinking can be a chemically gated gelation mechanism that is less sensitive to pH. An activation solution has been successfully used to form a radical crosslinking reaction, and these crosslinking reactions are suitable for use with acidic ablation compositions, as described further below. Chemical activation through pH gating of hydrogel crosslinking, as exemplified in the Examples, can provide a convenient approach to initiate and control the crosslinking process. Shear thinning materials such as hyaluronic acid gels can be potentially administered with alcohol loading, though they are less than ideal for this purpose since alcohols are not good solvents for hyaluronic acid and since the hydrogels formed can be squeezed out of the cervix by uterine contractions, thus presenting a safety concern due to potential exposure of adjacent tissues to high concentration alcohols.

Covalently crosslinked hydrogels are generally characterized by a hydrophilic core and functional groups that provide for the crosslinking. The precursors are generally water soluble, while the crosslinked polymers infuse with water but are insoluble due to their molecular weights resulting from extensive crosslinking into a hydrogel mass. The molecular weights of the precursor molecules and the number of functional groups influence the properties of the crosslinked hydrogel. In some embodiments, the chemical nature of the functional groups influences the crosslinking time, the nature of the accelerator composition, the predelivery stability, the biodegradation behavior, and other properties. Generally, the hydrogel design provides several parameters to influence the hydrogel properties.

As described herein, a hydrogel precursor formulation can be formed with high concentrations of alcohol without interfering with the hydrogel chemistry that allows for pH gated chemically driven crosslinking reactions. The precursor solutions are formulated to provide a buffer in an aqueous solution and hydrogel precursors in a solution primarily comprising alcohol solvent. As the hydrogel forms in situ, the alcohol from the precursor solutions is enveloped into the hydrogel mass. Contact between the hydrogel surface with the tissue delivers the alcohol to the tissue and results in the ablation.

Polyethyelene glycol based hydrogels have found diverse applications in medical contexts and provide the basis for several commercial FDA approved products such as Duraseal® and Duraseal® Exact (Integra Lifesystems), Mynx® (Cordis/Access Closure), SpaceOAR™ & SpaceOAR VUE™ (Boston Scientific) and Dextenza® (Ocular Therapeutix). In general, the crosslinking reactions to form the hydrogel can be based on various mechanisms, such as free radical polymerization processes or pH gated nucleophilic substitution. As demonstrated in the Examples, a convenient mechanism for crosslinking for the delivery of ablative alcohol makes use of addition reactions involving a nucleophilic group and an electrophilic group using pH to gate the crosslinking. The resulting hydrogels are generally bioresorbable, which may be based on gradual hydrolytic degradation.

### Hydrogels for Medical Applications

Hydrogels generally are considered materials that can absorb water, undergo swelling without discernible dissolution, and maintain three-dimensional networks capable of reversible deformation. See, e.g., Park, et al., Biodegradable Hydrogels for Drug Delivery, Technomic Pub. Co., Lancaster, PA (1993), incorporated herein by reference. Covalently crosslinked networks of hydrophilic polymers, including water-soluble polymers that are traditionally denoted as hydrogels (or aquagels) in the hydrated state. Hydrogels have been prepared based on crosslinked polymeric chains of methoxypoly(ethylene glycol) monomethacrylate having variable lengths of the polyoxyethylene side chains, and their interaction with blood components has been studied (Nagaoka et al., in Polymers as Biomaterial (Shalaby et al., Eds.) Plenum Press, 1983, p. 381).

Crosslinkable solutions for use include those that may be used to form implants in lumens or voids, and may form physical crosslinks, chemical crosslinks, or both. Physical crosslinks may result from complexation, hydrogen bonding, desolvation, Van der Waals interactions, ionic bonding, etc., and may be initiated by mixing two components that are physically separated until combined in situ or as a consequence of a prevalent condition in the physiological environment, such as temperature, pH, ionic strength, etc. Chemical crosslinking may be accomplished by any of a number of mechanisms, including free radical polymerization, condensation polymerization, anionic or cationic polymerization, step growth polymerization, etc. Where two solutions are employed, each solution contains components of a co-initiating system that initiate crosslinking on mixing. The solutions are separately stored and mixed for delivery into a tissue lumen. The improved hydrogel systems described herein are based on two solutions that mix to chemically crosslink.

Hydrogels may crosslink spontaneously from at least one precursor without requiring the use of a separate energy source. In the case of a dual-component system, mixing of the two solutions takes place so that the solutions are fluid while passing through the device. If desired, one or both crosslinkable precursor solutions may contain contrast agents or other means for visualizing the hydrogel implant. The crosslinkable solutions may contain a bioactive drug or other therapeutic compound that is entrapped in the resulting implant, so that the hydrogel implant serves a drug delivery function.

Properties of the hydrogel system, other than crosslinkability, can be selected according to the intended application. For example, if the hydrogel implant is to be used to temporarily fill and tamponade a reproductive organ, such as the uterine cavity, the hydrogel system can undergo some swelling to conform to irregular geometries and be biodegradable within the timeframe of a single menstrual cycle. Alternatively, degradation can be extended such that adhesion formation can be prevented long after delivery of an ablation agent has occurred. The hydrogel can be soft and have a modulus or stiffness that is lower than that of uterine tissue in a non-gravid uterus. Other applications may suggest different characteristics of the hydrogel system. More generally for medical applications, the materials should be selected on the basis of exhibited biocompatibility and lack of toxicity, both for the hydrogel and its degradation products. Even for ablation contexts, the degraded hydrogel should be non-toxic and biodegradable to avoid introducing systemic adverse effects due to clearance from the patient. The ablation compositions can be selected to have suitably low toxicity as it is highly diluted upon migration away from the treatment site.

A hydrogel may be biodegradable, so that it does not have to be retrieved from the body. Biodegradability, as used herein, refers to the predictable disintegration of the material into molecules or particles small enough to be metabolized or excreted under normal physiological conditions. Biodegradability may occur by, e.g., hydrolysis, enzymatic action, cell-mediated destruction, combinations thereof, or the like. With the use of biodegradable microparticles to aid in the delivery of the ablation compositions, the microparticles may degrade from the same mechanism or a different mechanism from an enveloping hydrogel.

Hydrogel Systems with Chemical Crosslinking

In embodiments of particular interest, the hydrogel systems described herein provide desirable space filling medical functions, especially for providing comprehensive surface area contact for ablation and, in some embodiments, reducing adhesions, can be provided in two solutions that are generally combined during delivery into the patient. The hydrogel precursors can be designed to spontaneously crosslink in the mixed solution based on a nucleophilic-electrophilic reaction or based on free radical polymerization, within a period of seconds. Through the appropriate design of the hydrogel systems, the precursors can be designed so that multiple precursors are combined into one solution that is stable against crosslinking, or the precursor solution can have a single precursor chemistry for radical crosslinking. A second solution can be provided that comprises an activator that initiates the crosslinking reaction following mixing.

Thus, a first aspect of the design of the polymer system is the formation of an appropriately stable initial formulation of the one or more precursors. While the polymer precursors should be appropriately stable to not significantly crosslink prior to delivery, the blended solutions of precursors and activator should result in rapid crosslinking. Due to these combinations of features, the configuration of the solutions for delivery can result in a hydrogel with slightly different properties relative to hydrogels formed with a different initial configuration in the solutions. The biodegradation rate is influenced by the incorporation of biodegradable linkages in the hydrogel. The chemical structures of the hydrogels and their precursors are described next in the context of these general properties, and the hydrogel properties are described in more detail below.

For crosslinking based on nucleophilic-electrophilic reactions, the precursors generally comprise at least two different polymerizable compounds. To form highly crosslinked hydrogels, both precursors generally have more than two reactive functional groups for forming crosslinks. In some embodiments, at least one of the precursor compounds is generally of moderate molecular weight and may be a polymer. Water soluble polymerizable polymeric monomers having a functionality>1 (i.e., that form crosslinked networks on polymerization) and that form hydrogels may be referred to herein as macromers. A second precursor compound can be a moderate molecular weight compound or a small molecule crosslinking agent. Molecular weight ranges are discussed further below. The functional groups of the precursors provide for the crosslinking reactions, the biodegradable feature as well as the overall properties of the precursor solutions and the product hydrogel. For crosslinking involving a free radiation reaction to form the hydrogel, a single hydrogel precursor can be used along with a separate activator solution. Prior to triggering free radical formation, the precursors can be stable even though the precursors ultimately react with equivalent monomers for crosslinking.

The crosslinking is generally triggered by some stimulus, such as energy, catalyst or reactant. Where two solutions are employed, each solution can contain one component of the polymerizing system that crosslink on contact, such that the crosslinking stimulus is the combing process itself. Other systems are known where energy, such as UV light, initiates crosslinking and acts as the stimulus. In the two-solution systems described herein, an activator or accelerator solution is used to trigger the crosslinking by changing the pH. The precursor are then mixed prior to delivery, and the mixed precursors are resistant to crosslinking until they are combined with the activator solution. For crosslinking based on radical reactions, an activator can comprise a free radical initiator, such as a peroxide.

The precursor solution with one or more precursor species and the activator solution can be stored in separate compartments of a delivery system and can be mixed in the delivery system when deposited on or within the tissue. Thus, the polymer systems exemplified herein are sufficiently stable that the two monomers are mixed in the delivery system prior to delivery and are combined with an initiating/activator solution upon delivery, where at least one of the monomers is usually a macromer. For exemplified embodiments, pH is used to gate the crosslinking reaction. Thus, the initiating/activator solution can raise the pH to allow for the crosslinking reactions to proceed, generally at a rapid rate. For free radical polymerization, the activator solution can trigger free radical formation to initiate crosslinking.

Using premixed precursors with an electrophile and nucleophile, the gel times can be very short, while still producing a uniform hydrogel with desirable properties. In contrast, if mixing and crosslinking occur simultaneously, there can be less homogeneity in the initial crosslinked material relative to a premixed precursor solution that is then mixed at delivery with an initialization solution that primarily shifts the pH, especially if the crosslinking times are especially short. The increased homogeneity improves the flow of the precursor solution leading up to the gel time where sufficient crosslinking effectively limits any further flow. After passing the gel time, further crosslinking occurs over an additional period of time until completion of the crosslinking. With free radical crosslinking, homogeneity is supported similarly by the initial mixture of precursors.

In hydrogel systems based on nucleophilic-electrophilic addition reactions, suitable functional groups for crosslinking macromers to form tissue implants in situ also may be advantageously used, including macromers that contain groups that demonstrate activity towards functional groups such as amines which may be present in the crosslinking compound. Thus, multi-component hydrogel systems can spontaneously crosslink when the components are activated by an activating systems, but the components, such as two or more components, are appropriately stable for a reasonable process time before activation. Such systems include, for example, monomers (macromers or small molecule) that are di or multifunctional amines in one component and macromers with di or multifunctional N-succinimidyl containing moieties in the other component. Succinimidyl functional groups facilitate amide bond formation in reactions with amines. Other activator systems, such as components of redox type activators, also may be used with precursors amenable to free radical polymerization, such as acrylate functional groups or the like. The mixing of the two or more solutions may result in a condensation polymerization that further leads to the formation of a hydrogel.

The solids concentration of the hydrogel precursor solution has been discovered to be significant for intrauterine delivery. The intrauterine space can contain various bodily fluids, and after medical procedures, blood and residual fluid from the procedure can also be present. While some fluid may be removed prior to instillation of the hydrogel into the intrauterine space, the hydrogel precursors can be diluted by the fluids in the space where the precursors are delivered. The improved hydrogels described herein are designed to be dilution resistant. Free radical crosslinkable hydrogels with good dilution resistance have also been described, see published U.S. patent application 2019/0351107 to Sawhney et al. (hereinafter the '107 application), entitled “Embolic Compositions and Methods,” incorporated herein by reference.

Hydrogel and precursor solution properties are described in detail below. Significant properties include: gel time, opportunity for appropriately large alcohol (or other cytotoxin) volume percent, biocompatability, lack of clogging of applicator, low dilution effects/good spread through the cavity without excessive migration, appropriate biodegradation times, and maintenance of sufficient modulus for a desired period of time. Due to muscular function, the uterus can expel materials from within the cavity, so a modulus below certain values could be subject to expulsion from the uterine cavity before the intended therapeutic effect has bene achieved. The parameters that influence these issues include: functional group chemistry, crosslinking density/molecular weights of the monomers, monomer composition, percent solids in the hydrogel precursors, and activator solution properties, such as buffer chemistry and ionic strength.

Polyethylene glycol based hydrogels have generally been designed for biodegradation though hydrolysis. The hydrolysis reactions can be sensitive to pH. For nucleophilic-electrophilic addition reactions, through the use of premixed precursors with acidified amine groups, the autohydrolysis of the product hydrogel may be observed. Autohydrolysis can result in degradation of the hydrogel without effects of biological activity. Autohydrolysis can take place in the initially formed hydrogel prior to full removal of the buffer from the hydrogel through osmosis and exchange with bodily fluids. Autohydrolysis can result in premature decrease in the modulus of the hydrogel. With the hydrogels formed as described herein, any autohydrolysis is within acceptable limits.

For the delivery of an acidic ablation composition, such as acetic acid, the acid can prevent the use of a pH gated crosslinking reaction. As described further below, free radical polymerization is generally not pH sensitive, so crosslinking can be initiated in the presence of acids. The presence of the acids though may increase the rate of hydrolysis and thus degrade the hydrogels more quickly. With the selection of appropriate hydrogel functional groups, the degradation time may still be acceptable to perform the desired ablation. In alternative embodiments, an acid can be delivered in a different phase, such as microparticles, that release the ablation compositions gradually to reduce the hydrolysis rate. The microparticles can release the ablation composition through elution and/or through biodegradation of the microparticles.

### Monomers

Monomers capable of being crosslinked to form a biocompatible implant may be used. As noted above, monomers can be macromers or small molecule crosslinking agents and in either case may or may not be polymers. The term polymer, as used herein, means a molecule formed of at least three repeating groups, which may have pendent functional groups. The term “reactive precursor species” means a polymer, functional polymer, macromolecule, small molecule, or small molecule crosslinker that can take part in a reaction to form a network of crosslinked molecules, e.g., a hydrogel.

Monomers may include the biodegradable, water-soluble macromers described in U.S. Pat. No. 7,332,566 to Pathak et al. (hereinafter the '566 patent), entitled “Biocompatible Crosslinked Polymers With Visualization Agents,” incorporated herein by reference. These monomers are characterized by having at least two polymerizable groups, separated by at least one degradable region. When polymerized in water, they form coherent gels that persist until eliminated by bio-degradation. Generally, the macromer is formed with a core of a polymer that is water soluble and biocompatible, such as a polyalkylene oxide, e.g. polyethylene glycol, which can be flanked by hydroxy-carboxylic acids such as lactic acid. Suitable monomers, in addition to being biodegradable, biocompatible, and non-toxic, also can be at least somewhat elastic after crosslinking or curing. Crosslinkable monomers with amine groups include, for example, polyethylenimine, which is soluble in alcohol and can have selectable degrees of branching and molecular weights, or branched polyethylene glycol with amine terminal functional groups.

The macromers generally have biologically inert and water soluble cores. When the core is a polymeric region that is water soluble, polymers that may be used include: polyethers, for example, polyalkylene oxides such as polyethylene glycol(“PEG”), polyethylene oxide (“PEO”), polyethylene oxide-co-polypropylene oxide (“PPO”), co-polyethylene oxide block or random copolymers, poloxamers, such as Pluronic® F-127; as well as polyvinyl alcohol (“PVA”); poly (vinyl pyrrolidone) (“PVP”); poly (amino acids); polysaccharides, such as hyaluronic acid or cellulose and derivatives thereof; dextran and proteins such as albumin. Based on extensive experience in existing medical products, polyethers and more particularly poly(oxyalkylenes) or poly(ethylene glycol) are especially suitable. For the applications herein, the polymeric regions should also be soluble in high alcohol concentrations, and monomer cores comprising PEG, PEO, poloxymer, PVP, and copolymers thereof may generally be appropriate. Hydrogels have also been described based on polyoxazolene copolymers. See, published U.S. patent application 2019/0231923 to Hoogenboom et al., entitled “Cross-Linked Polymers and Implants Derived From Electrophilically Activated Polyoxazoline,” incorporated herein by reference. Macromer cores based on polyoxazolene can be similarly used.

It has been determined that hydrogels formed with macromers with longer distances between crosslinks are generally softer, more compliant, and more clastic. Thus, in the polymers of the '566 patent, increased length of the water-soluble segment, such as polyethylene glycol, tends to enhance elasticity. Molecular weights of hydrophilic macromers, such as macromers with polyethylene glycol cores, for desirable applications can be from about 5,000 to about 500,000, in further embodiments from about 7500 to about 100,000, in some embodiments from about 10,000 to about 50,000, and in other embodiments in the range of about 15,000 to about 40,000, as well as corresponding ranges with interchanged upper and lower boundaries, such as about 5,000 to about 40,000. As used herein, molecular weights (mass) are in conventional units, which can be equivalently Daltons or as a molar mass-grams/mole (assuming natural isotopic presence in either case), and for polymers molecular weights are generally reported as averages if there is any distribution of molecular weights. A person of ordinary skill in the art will recognize that additional ranges within the explicit ranges above are contemplated and are in the present disclosure.

For crosslinking based on nucleophilic-electrophilic reactions, the monomer providing electrophilic functional groups generally is a macromer. The monomer providing the amine functional groups can be a macromer or a small molecule crosslinker. Macromers with amine functional groups can fall within the molecular weight ranges of the previous paragraph for the macromers with electrophilic functional groups. While somewhat arbitrary, small molecule crosslinkers are identified herein as having a molecular weight of no more than 4500 and can be considered macromers if the molecular weight is greater than 4500. Thus, small molecular crosslinkers can have molecular weights generally from 100 to about 4500, in further embodiments, from about 200 to about 2500, and in additional embodiments form about 225 to about 1500, as well as comparable ranges with any upper and lower limit from these ranges, such as from about 100 to about 1500. A person of ordinary skill in the art will recognize that additional ranges of crosslinker molecular weights within the explicit ranges above are contemplated and are within the present disclosure. When the core is small molecular in nature, any of a variety of hydrophilic functionalities can be used to make the precursor water soluble. For example, functional groups like hydroxyl, amine, sulfonate and carboxylate, which are water soluble, maybe used to make the precursor water soluble. In addition, N-hydroxysuccinimide (“NHS”) ester of subaric acid is insoluble in water, but by adding a sulfonate group to the succinimide ring, the NHS ester of subaric acid may be made water soluble, without significantly affecting its reactivity towards amine groups.

For free radical polymerization, a single precursor can be used since the radical initiated addition reaction can be based on combining two like functional groups. The precursor molecules are generally macromers to provide appropriate hydrogel modulus values of relevance for many applications. Appropriate molecular weight ranges would be the same as described two paragraphs above for hydrophilic macromers for nucleophilic-electrophilic addition reactions, and these molecular weight ranges can be considered as explicitly written in this paragraph.

### Biodegradable Linkages

If it is desired that the biocompatible crosslinked hydrogel polymer be biodegradable or absorbable, one or more precursors having biodegradable linkages present in between the functional groups may be used. The biodegradable linkage optionally also may serve as part of the water soluble core of one or more of the precursors. In the alternative, or in addition, the functional groups of the precursors may be chosen such that the product of the reaction between them results in a biodegradable linkage. For each approach, biodegradable linkages may be chosen such that the resulting biodegradable biocompatible crosslinked polymer degrades or is absorbed in a desired period of time. Generally, biodegradable linkages are selected that degrade under physiological conditions into non-toxic products and maintain the overall mechanical strength of the hydrogel for such time that the implant resists expulsion from the uterine cavity or other implantation site.

The biodegradable linkage may be chemically or enzymatically hydrolyzable or absorbable. Illustrative enzymatically hydrolyzable biodegradable linkages include peptidic linkages cleavable by metalloproteinases and collagenases. Additional illustrative biodegradable linkages can be functional groups on the core polymers and copolymers, such as hydroxy-carboxylic acids, orthocarbonates, anhydrides, lactones, (aminoacids, carbonates, phosphonates or combinations thereof. In exemplified embodiments, the biodegradable linkages are ester (hydroxy-carboxylic acid) moieties adjacent the electrophilic group used for crosslinking. These linkages hydrolyze without enzymatic activity under physiological conditions. Thus, biodegradable broadly refers to gradual degradation under physiological conditions regardless of the mechanism.

As described further below, loaded microparticles used for ablation chemical delivery can comprise water insoluble biodegradable polymers. The availability of suitable biodegradable, non-hydrogel polymers for reservoirs of the ablation compositions provides flexibility for delivery under desirable degradation conditions. Some polymers may degrade by hydrolysis, such as polylactic acid-co-glycolic acid (PLGA), while other may degrade by enzymatic pathways, such as polycarbonates. Acids, such as acetic acid or trichloroacetic acid, delivered as an ablation agent may shorten times for hydrolytic degradation. Shortened times for hydrolysis due to an acid may still provide for suitable times for completion of the ablation, but the use of appropriately selected microparticles can provide an extra degree of control for the hydrogel delivery of the ablation composition. Use of microparticles and the like are described further below.

For biodegradable linkages that hydrolyze under aqueous conditions, the presence of acidic ablation compounds can alter the degradation rate, generally speeding the degradation. In general, the degradation rate can be influenced by the selection of the particular functional groups. For example, the particular ester groups connected to a hydrophilic core can influence the rate of hydrolysis. Depending on the specific acid and particular degradable moieties, the degradation rate may or may not be within a target range. The ester can be selected to slow the hydrolysis in the presence of an acid ablation composition. Sequestering the ablation composition into microparticles or similar additional phase, can provide an extra degree of control on the degradation rate. Microparticles or the like can be selected to have a hydrolysis rate sufficiently slow to provide suitable degradation times in the presence of an acid, or microparticles can be selected to degrade based on enzymatic cleavage, so that the acid has little or no effect on the degradation rate. Thus, the use of biodegradable microparticles or the like provide an additional layer of system design options to achieve desired results.

### Functional Groups, Precursor Solutions and Crosslinking Reactions

The crosslinking reactions generally are designed to occur in aqueous solution in vivo, encircled by physiological conditions, where the hydrogel reaction occurs in a transient local environment, which, for example, may have a pH significantly higher than physiological. While hydrogel crosslinking can be based on pH triggered electrophile-nucleophile crosslinking reactions, alternative reactions can be performed using free radical polymerization. Radical crosslinking can be initiated with radiation, but radical reactions can be conveniently initiated with an activator solution. Thus, for either electrophilic-nucleophilic crosslinking or free radical crosslinking, the crosslinking reactions occur “in situ”, meaning they occur at local sites such as on organs or tissues in a living animal or human body. Due to the in situ nature of the reaction, the crosslinking reactions can be designed not to release undesirable amounts of heat of polymerization. Crosslinking times for desirable procedures are described above. As noted further below, the precursor solutions can be formed with solvent that primarily comprises an ablation composition, such as an alcohol, in an aqueous solution with appropriately selected amounts of water and/or other solvent components. In some embodiments, a lower amount or no ablation composition can be included in the precursor solution if some or all of the ablation composition can be delivered with the activator solution. The target concentration of the ablation composition within the formed hydrogel influences the amounts of ablation composition within the solutions combined to form the loaded hydrogel.

With respect to addition reactions resulting from nucleophilic-electrophilic reactions, certain functional groups, such as alcohols or carboxylic acids, do not normally react with other functional groups, such as amines, under physiologically acceptable pH (e.g., pH 7.2-11.0, 37° C.). However, such functional groups can be made more reactive by using an activating group such as N-hydroxysuccinimide or derivatives thereof. Several methods for activating such functional groups are known in the art. Suitable activating groups include carbonyldiimidazole, sulfonyl chloride, aryl halides, sulfosuccinimidyl esters, N-hydroxysuccinimidyl ester (NHS), succinimidyl ester, epoxide, aldehyde, maleimides, imidoesters and the like. The N-hydroxysuccinimide esters or N-hydroxysulfosuccinimide groups are desirable groups for crosslinking of proteins or amine functionalized polymers such as amino terminated polyethylene glycol (“APEG”) since they have found acceptance in medical implants from long periods of use in approved products. The activated ester groups for supplying electrophiles for crosslinking can be similarly grafted onto other hydrophilic polymer cores, such as polyoxazolene copolymers.

For embodiments based on electrophile-nucleophile based crosslinking, the other functional group used for the crosslinking generally is an amine. Amines are weak bases, and the pKa of the protonated amines are dependent on the molecule. The protonated amines are generally not suitable for nucleophilic substitution. Therefore, the precursors generally have the nucleophile comprising an acidified amine precursor (—NH₃⁺ A⁻, where A is anion that is a conjugate base of a strong acid, such as a halide ion, e.g., Cl⁻), which can be referred to in the art as a salted amine. Thus, the precursor solution can be at a suitable pH to maintain substantially protonated amines prior to combining with the activator solution. This pH adjustment can maintain the precursor solutions from prematurely crosslinking, and using a salted amine avoid rapid crosslinking prior to acidification of the amine in the mixed solution. Suitable small molecule amine precursors include lysine molecules and oligomers thereof. Specific small molecule amines include, for example, lysine, dilysine, trilysine, tetralysine, pentalysine, and mixtures thereof. The lysine amines are generally protonated under physiological (close to neutral) pH values. The use of a small molecule crosslinker for one precursor can provide for a relatively low viscosity of the blended precursors prior to extensive crosslinking such that the blended hydrogel precursors can be delivered through a thin catheter while crosslinking initiates and the fluid conforms to the shape of the uterine cavity, but then relatively rapid crosslinking in the presence of the accelerator provides for stabilization of the hydrogel within the uterus in a reasonable period of time. In the hydrogels formed as described herein, the precursors are preblended, and the amines are provided in a protonated form. Macromolecular amines function well for rapid crosslinking in the premixed systems.

While the mixed precursors are reasonably stable, their stability times are not long in the sense of product distribution. Thus, aqueous solutions of NHS based crosslinkers and functional polymers can be made just before the crosslinking reaction. For example, two separate vials of the electrophile and the nucleophile can be combined and placed into a syringe tube along with a solution comprising the ablation composition. The expression “pot life” can refer to the time from mixing. Using similar precursors solutions in aqueous solutions without an ablation composition, it was observed that longer “pot life” may be obtained by keeping the solutions at lower pH. Generally, the pre-mixed precursors can be kept as a pH from about 3.5 to about 6.5, in some embodiments from about 3.75 to about 6.3 and in further embodiments from about 4 to about 6.2 pH units. With high alcohol concentration precursor solutions, suitable pot life and gelation properties have been obtained without a buffer in the precursor solution. In other embodiments, it may be desirable to have an optional buffer. In appropriate embodiments, the precursor solutions can be buffered to maintain the solutions at the appropriate pH values until mixed with the activator solution. Suitable buffers include, for example, phosphate buffer and/or citrate buffer. Suitable buffers may include biological buffers, for example, as sold by Sigma Aldrich (https://www.sigmaaldrich.com/US/en/technical-documents/protocol/protein-biology/protein-concentration-and-buffer-exchange/buffer-reference-center), The precursor solution can have a total buffer salt concentration from about 2 mM (millimolar) to about 500 mM, in further embodiments from about 5 mM to about 300 mM, in other embodiments from about 7 mM to about 150 mM, and in some embodiments from about 10 mM to about 100 mM. Adjustment of the pH can be accomplished with appropriate addition of a strong acid or base to get a target pH.

As the precursor blend flows across the tissue, the hydrogel forming during the crosslinking process can conform to the shape of the small features of the tissue such as bumps, crevices and any deviation from surface smoothness, although perfect conformation is not necessary. Also, precursor flow can surround growths, such as fibroids, such that the fibroids do not block the flow to contraindicate the procedures, while providing the added benefit that the ablation treatment can also ablate the fibroid. Fibroid ablation can remove the motivation for a hysterectomy. In contrast, the presence of fibroids can contraindicate some ablation procedures based on energy sources. In other contexts, the chemical ablation can serve as an alternative to radiation based fibroid ablation. RF ablation for fibroids that are not too large using the Hologic® Acessa® Laparoscopic Radiofrequency Ablation system is based on a laparoscopic procedure involving small abdominal incisions, which are unnecessary for the transcervical approach.

The crosslinking density of the resultant biocompatible crosslinked polymer is controlled by the overall molecular weight of the crosslinker, if used, and functional polymer (macromer) and the number of functional groups available per molecule for crosslinking. A lower molecular weight between crosslinks such as 600 Da will give much higher crosslinking density as compared to a higher molecular weight such as 10,000 Da. Generally, the molecular weights between crosslinks can range from about 100 Da to about 40,000 Da, in further embodiments form about 250 Da to about 20,000 Da, and in other embodiments from about 400 Da to about 12,000 Da. A person of ordinary skill in the art will recognize that additional ranges of molecular weights between crosslinks within the explicit ranges above are contemplated and are within the present disclosure. Higher molecular weight functional polymers can be used to obtain more elastic gels.

The crosslinking density also may be controlled by the overall percent solids of the precursor solution, i.e. functional polymer (macromer) solutions with an optional crosslinker. Increasing the percent solids increases the probability that an addition reaction, such as an electrophilic functional group combining with a nucleophilic functional group, occurs prior to inactivation by hydrolysis. Yet another method to control crosslink density for nucleophilic-electrophilic addition, is by adjusting the stoichiometry of nucleophilic functional groups to electrophilic functional groups. A one to one ratio leads to the highest crosslink density. In general, over time, the hydrogel completes curing so that available crosslinking sites form crosslinking bonds. If the electrophilic and nucleophilic compounds are provided in equal or equivalent amounts it can be expected that approximately all functional groups form crosslinking bonds after full curing. Equal numbers (or reaction equivalents) of the two types of agents provides the highest crosslinking density. If a different ratio of functional groups is used, the properties of the cured hydrogel can be accordingly somewhat different. The crosslinking density can be dependent on the number of functional groups on the precursor molecules as well as the ratio of precursor molecules. A non-stoichiometric ratio of electrophilic and nucleophilic groups can be used to alter the crosslinking density if desired. In some embodiments, the ratio of electrophilic functional groups to nucleophilic functional groups can be from 0.8:1.0 to 1.0:0.8. A person of ordinary skill in the art will recognize that additional ranges within these explicit ranges are contemplated and are within the present disclosure.

The solid contents of the blended precursor solutions can influence the properties of the resulting hydrogel as well as the ability of the precursor solution to fill the target space prior to crosslinking. If the solid content is too low, the hydrogel can be more susceptible to dilution effects from fluid present in the space at the time of delivery of the precursor solution. Even if the space is drained, there often can be significant amounts of residual fluid present. Dilution effects can inhibit desired space filling, can lead to irreproducible results, and can result in incomplete crosslinking that results in a low modulus hydrogel implant. Excessively high solid content can result in a precursor solution that crosslinks too quickly and that has a viscosity that is higher than desired, which can result in clogging of the applicator and/or incomplete filling of the targeted space. Note that the viscosity changes quickly due to the fast gelation times, and once gelled, the viscosity is essentially infinite. Generally, the blended precursor solution delivered from the applicator has a solid content from about 3 weight percent (wt %) to about 20 wt %, in further embodiments from about 3.5 wt % to about 18 wt %, and in other embodiments from about 3.75 wt % to about 16 wt %. A person of ordinary skill in the art will recognize that additional ranges within the explicit ranges above are contemplated and are within the present disclosure.

For the macromers for crosslinking, including electrophilic macromers or macromers with amine groups or macromers with free radical polymerizable groups, the polymers can have a plurality of arms each with a terminal functional group suitable for crosslinking. As noted above polyethylene glycol (PEG) based macromers are established hydrogel precursors and core compounds are commercially available. PEG is also known in the art as polyethylene oxide (PEO), and these names are interchangeable. The PEG cores can have 3, 4, 5, 6, 7 8, 9, 10 or more arms and 4 arm (4A) and 8 arm (8A) PEG are convenient degrees of branching. The macromers can also have selected molecular weights, and suitable ranges can be from about 5,000 Da (10 kDa) to about 100,000 Da (100 kDa), in further embodiments from about 7,500 Da to about 75,000 Da, and in other embodiments from about 10,000 Da to about 60,000 Da. A person of ordinary skill in the art will recognize that additional ranges of macromer molecular weights and various ranges of macromer arms from the specific values within the explicit ranges or values above are contemplated and are within the present disclosure.

In general, the precursors (e.g., electrophiles and/or nucleophiles) are generally shipped in sterile vials. At an appropriate point in a procedure, such as at the beginning of the procedure, these can be mixed with solvent and loaded into a syringe. Various configurations of the vials and other sterile components can be provided to facilitate this processing. Depending on the anticipated time of use, the pot life can be appropriately tracked.

For radical based polymerization, the polymer cores and branching can be similarly selected to achieve crosslinked hydrogel properties. For these precursors, a single precursor can be used with an appropriate functional group to be activated for radical polymerization/crosslinking. For the crosslinking reactions of particular interest herein, free-radical polymerizable group can comprise an unsaturation such as an unsaturated hydrocarbon group, e.g., a vinyl group. During free-radical polymerization, monomers can be successively added to a growing chain. An initiated free radical has a reactive species that adds to other monomer unit to grow the polymer chain while maintaining the radical until it is terminated. To initiate the reactions, free radicals can be formed in response to activators, which are discussed further in the next section.

Monomers can be macromers with hydrophilic cores with pendent functional groups with an unsaturated hydrocarbon, such as a vinyl group (—CH═CH₂). Vinyl groups may be used as functional groups on a precursor, e.g., a polymer may be derivatized to carry a vinylic functional group. Vinylic functional groups include, for example, acrylate groups or derivatives thereof with various alkyl groups bound to the ester oxygen, such as a methyl group. The term group refers to a chemical moiety that may be substituted, and the substituents may, in turn, be substituted. Derivatives of an acrylate group include a methacrylate group.

### Activator Solution

An activator solution can provide for triggering the crosslinking reaction. Two or more solutions can be combined to start the crosslinking reaction during delivery of the hydrogel to the target location. The activator solution can trigger a pH change or initiate a free radical reaction. Depending on the composition of the ablation composition (cytotoxic agent), the ablative composition may be delivered with a precursor solution and/or an accelerator/activator solution. The volume of activator solution is generally equal to or less than the volume of the precursor solution since the volume of the precursor solution should allow for a concentration of precursors that is not too large. If the ablation composition is in only one of the two solutions, such as only in the precursor solution, then it can be desirable for the volume of the activator solution to be less than the precursor solution to avoid excessive dilution of the ablation composition. In particular, for acetic acid based ablation compositions, it may be suitable to put the ablation composition in the activator solution or in both the activator solution and the precursor solution. For alcohol ablation composition, desirable results have been obtained with placement of the alcohol in the precursor solution, but at least some alcohol may be placed in the activator solution.

In embodiments based on crosslinking with an electrophilic agent and a nucleophilic agent, the crosslinking reaction might occur slowly under neutral conditions, but the addition of a pH based activator, such as a basic buffer, accelerates the reaction such that it occurs over a desirable time frame. Thus, for the hydrogels of particular interest herein, suitable buffers are near a neutral pH although somewhat basic or alkaline, and can include, for example, borate, phosphate, citrate, bicarbonate, CHES (N-Cyclohexyl-2-aminocthanesulfonic acid), TAPS ([tris(hydroxymethyl)methylamino] propanesulfonic acid), Bicine (2-(bis(2-hydroxyethyl)amino)acetic acid), Tris (tris(hydroxymethyl)aminomethane), Tricine (N-[tris(hydroxymethyl)methyl]glycine), CAPS (N-cyclohexyl-3-aminopropanesulfonic acid), CABS (C₁₀H₂₁NO₃S), CAPSO (C₉H₁₉NO₄S), or the like. Some of the buffer salts can be provided in hydrated forms. The selected hydrogel precursors can be initially mixed to have a pH near neutral to slightly acidic to provide for slow crosslinking until mixed with the activator buffer.

Mixed buffers can be particularly desirable for activator solutions. Mixed buffers are known to extend the range of buffer capacity relative to the individual buffers. The mixed buffers for the activator solutions are able to provide desired basic pH values but then transition to more neutral pH values following completion of the crosslinking. As used herein and generally in this art, the terms basic and alkaline are used interchangeably for reference to solutions with higher pH values. So the pH is maintained to complete the crosslinking, but with moderate buffer capacity. Generally, one of the buffers in the mixture has a relatively high pKa, e.g., above 9, such as borate or CHES, while another buffer has a more neutral pH, such as phosphate or citrate. The relative amounts of the two or more buffers are not particularly limited as long as the desired pH values are achieved. Total buffer concentration ranges for some embodiments are given below. In some embodiments, the weight ratio of the more neutral buffer to the more alkaline buffer can be from about 0.05 to about 15, in further embodiments from about 0.2 to about 12 and in some embodiments from about 0.25 to about 10. A person of ordinary skill in the art will recognize that additional ranges within the explicit ranges are contemplated and are within the present disclosure.

For free-radical driven crosslinking with in vitro polymer crosslinking, activators can be used that are thermally activated or radiation activated, such as with dicumyl peroxide (a thermally unstable organic peroxide) or commercially available UV or visible light initiated radiation initiators. While these approaches can be used, thermal or radiation activation approaches are less convenient for in situ activation during delivery into a patient. Chemical activation can be effectively used, for example, with an activator and a co-activator for radical initiation.

Peroxides combined with a metal reductant can be suitable for in vivo use for free radical initiation. For example, iron +2 salts can supply a biocompatible reactant that reacts with peroxides to generate OH^{·} radicals to initiate the crosslinking reactions. The reductant, such as a suitable metal ion, can be termed a co-initiator and would be kept separate from the initiator (such as a peroxide) until initiating the crosslinking. In general, the initiator and co-initiator can be combined into the precursor solution, but storage stability may suggest a particular combination. Suitable metal ions as reducing agents for a peroxide include, for example, Fe2+, Cr2+, V2+, Ti3+, Co2+, and Cu+. These may be provided as a suitable salt, such as ferrous sulfate, ferrous lactate, ferrous gluconate, a copper salt or a combination thereof. Salts may comprise a biocompatible anion, such as sulfates, chlorides, carbonates, or the like. A concentration of a reductant ion in an activation solution can be from about 0.2 to about 200 mM, in further embodiments from about 1 mM to about 150 mM and in further embodiments from about 5 mM to about 100 mM. A person of ordinary skill in the art will realize that additional ranges of reducing agent concentration within the explicit ranges above are contemplated and are within the present disclosure.

Peroxides for use as initiators can be organic peroxides and/or inorganic peroxides. Organic peroxides are organic compounds containing a peroxide functional group (ROOR′). If the R′ is hydrogen, the compounds are called organic hydroperoxides. Peresters have general structure RC(O)OOR. Organic peroxides can be divided into classes such as peroxyesters, peroxy(di)carbonates, diacyl peroxides, dialkyl peroxides, peroxyketals and hydroperoxides, e.g., tertbutyl hydroperoxide or tert-amyl hydroperoxide. The O—O bond easily breaks, producing free radicals of the form RO·. Hydrogen peroxide (HOOH) is a well-known inorganic peroxide. Other inorganic peroxides include, for example, persulfates, such as sodium persulfate, ammonium persulfate, potassium persulfate or mixtures thereof. Peroxide concentrations in an initiator solution can be from about 50 to about 20,000 parts per million (ppm) by weight; in further embodiments from about 100 ppm to about 10,000 ppm and in additional embodiments from about 200 ppm to about 5000 ppm. A person of ordinary skill in the art will recognize that additional ranges of peroxide concentrations within the explicit ranges above are contemplated and are within the present disclosure.

### Hydrogels Based on Electrophile-Nucleophile Crosslinking for Chemical Ablation

While other nucleophilic groups can be used, amines have been found to provide desirable performance. A polyamine can provide desired crosslinking in the resulting polymer. Polylysines, such as trilysine, have been used in forming medical hydrogels, and trilysine is used in one example below. However, lysine is not very soluble in alcohol, so somewhat lower alcohol concentrations would generally be used in the precursor solutions if trilysine is a reactant. Polyethylene glycol amines though can work well in alcohol solutions, and these precursors are appropriate for use in forming medical hydrogels. As demonstrated in the examples, polyethylene glycol based hydrogels can be adapted for forming hydrogels for ablation with alcohol up to high levels in the precursor solutions. The amines can be provided in protonated form based on acidification with an appropriate acid to form a salt having R—NH₃⁺ A moieties, where A is the conjugate base of the acid. In precursor solutions with solvents having high alcohol concentrations, the amines remain protonated without adding buffer to the solvent. As demonstrated in the examples below, the amines remain sufficiently protonated to block crosslinking reactions to provide appropriate pot lives for the solutions. Buffers may not be readily soluble in the high alcohol solvents, so the buffers can be delivered in a separate aqueous accelerator solution with the precursors blended in a precursor solution with a high alcohol content. In contrast, corresponding aqueous solutions in the '276 application cited above used buffer in the precursor solution to provide for desired stability.

The use of hydrogels for intrauterine use was described in published U.S. patent application 2005/0266086 to Sawhney (hereinafter the '086 application), entitled “Intrauterine Applications of Materials Formed In Situ,” incorporated herein by reference. A product called SprayGel™ was sold generally for prevention of adhesions based on polyethylene glycol hydrogels. The '086 application specifically describes the use of SprayGel™ in its Examples. The product in SprayGel™ was covered under U.S. Pat. No. 7,009,034 to Pathak et al. (hereinafter the '034 patent), entitled “Biocompatible Crosslinked Polymers,” incorporated herein by reference. An improved hydrogel for intrauterine use is described in the '276 application cited above.

The polyethylene glycol precursors can be designed to achieve desired hydrogel properties within reasonable ranges with respect to alcohol loading and hydrogel parameters discussed in the following. For ablation applications, a fast crosslinking time is generally desirable and various other parameters may be selected as desired. Relevant parameters adjustable by precursor engineering include, for example, gel times, degradation times, crosslinking density, swelling, hydrogel elasticity and density, elution times, and the like. The delivery approach provides for forming a uniform hydrogel with very short gel times through the premixing of the precursors.

As described further below, the hydrogel precursors generally comprise a multivalent electrophilic precursor with three or more electrophile groups and a multivalent nucleophilic precursor with three or more nucleophile groups to provide formation of an appropriately crosslinked hydrogel. The precursor molecular weights and the number of crosslinking groups influence the hydrogel density and swelling. The nucleophilic group is generally an amine. An addition reaction between the nucleophile and the electrophile can be triggered by a pH change. The precursors can be premixed for delivery to the patient to produce a more uniform hydrogel. Since the hydrogels with the alcohol can be designed for very rapid gelation, the premixing is again desirable for produce a uniform hydrogel with good mechanical properties. The stability of the mixed precursors for a reasonable period of time can be accomplished by protonating the amine with a suitable acid since the acidified amine is then reversibly blocked from acting as a nucleophile. An activation liquid is mixed with the precursors during delivery to raise the pH and deprotonate the amines to drive the crosslinking reaction. Since the precursors are premixed, the crosslinking can form a uniform hydrogel without needing to uniformly mix the precursors and the activator solution, allowing for very rapid gel times.

While in general, the two hydrogel precursors can be delivered in separate solutions and combined during the delivery process, it has been found that combining the precursors in a precursor solution well mixed prior to delivery is conducive to the achievement of good hydrogel properties while targeting very short gel time. A second solution, which can be referred to as an activator solution, provides a high pH buffer that is combined with the precursor solution during delivery. The activator solution can comprise a buffer solution having a pH greater than 8.2 and a sufficient buffer capacity to raise the pH of a mixture of the precursor blend solution and the accelerator solution above a pH of 8. The high pH solution deprotonates the amine so that it can participate in the crosslinking reactions. With this approach, very fast gel times can be achieved while obtaining a uniform hydrogel with desired mechanical properties. Suitable activator solutions are described above, and specific buffers are further described in the examples.

The activator solution is generally aqueous to dissolve the buffers. Therefore, the precursor solution provides the alcohol or other high volume ablation composition for the hydrogel. To achieve the desired amounts of alcohol or other ablation composition in the hydrogel, it is generally desirable to have a larger volume of the precursor solution relative to the activator solution. The appropriate relative volumes are influenced by the target alcohol or other ablation composition concentration and the solvent in the precursor solution. While the precursor solutions may comprise only alcohol solvent, some water may be included to improve solubility. In some embodiments with the precursor composition in the precursor solution, the precursor solution has from about 0 to about 30 volume percent (vol %) water, in some embodiments from about 1 vol % to about 25 vol % water, in further embodiments from about 2.5 vol % to about 20 vol % water, and in other embodiments from about 5 vol % to about 15 vol % water, with the balance generally being alcohol or other ablation liquid with the ablation composition. Some precursors may not be soluble in pure alcohol, so even a smaller amount of water can significantly facilitate solubilizing some precursors, such as compounds with acidified amines. A person of ordinary skill in the art will recognize that additional ranges of water concentrations within the explicit ranges above are contemplated and are within the present disclosure.

The solvent amounts can be evaluated based on solvent volumes provided to the solutions. For example, if the precursor solutions has X vol % water and 100−X vol % alcohol, where X is within the ranges above, the hydrogel initially has ((X×M)+N) vol % water and (100−X)×M vol % alcohol, where M is the volume of precursor solution divided by the total volume of the precursor solution plus the activator solution and N is the volume of the activator solution divided by the total volume of the precursor solution plus the activator solution. The relative volumes (or volume ratio) of the precursor solution to the activator solution can be from about 1:1 to about 25:1, in some embodiments from about 1.25:1 to about 20:1, in further embodiments from about 1.5:1 to about 15:1 and in other embodiments from about 2:1 to about 10:1 ratio of precursor solution volume to activator solution volume. The resulting volume percent of alcohol relative to total solvent in the hydrogel at the start of gelation is from about 50 vol % to about 95 vol %, in further embodiments from about 55 vol % to about 92.5 vol % and in other embodiments from about 60 vol % to about 90 vol %. A person of ordinary skill in the art will recognize that additional ranges of percent volume water in precursor solution, relative solution ratios and volume percent of alcohol in the hydrogels within the explicit ranges above are contemplated and are within the present disclosure.

Gel times may be varied by altering the pH, temperature, and buffer salt strength of the “activator” portion of the in situ system. In one embodiment, the pH of the activator solution used to initiate crosslinking in the system is in a pH range from about 7 to 12.5. In further embodiments, the pH range of the activator solution is from about 7.5 to about 12, in some embodiments, in a pH range from about 8 to about 11.75, and in additional embodiments, in a pH range from about 8.5 to about 11.25. In one embodiment, the total activator buffer salt concentration ranges from about 25 mM to about 1.5M. In some embodiments, the buffer salt concentration is from about 50 mM to about 1M, and in further embodiments from about 100 mM to 700 mM. As noted above, this total buffer concentration can be divided between two or more different buffers to obtain a desired buffering range. A person of ordinary skill in the art will recognize that additional ranges of pH and concentration within the specific ranges above are contemplated and are within the present disclosure. The selected pH and buffer concentration of the accelerator solution can be influenced by the dilution ratio with the precursor solution and the desired gel time.

When delivered into the uterine cavity, gelation evaluation can be obfuscated somewhat by delivery of fresh precursors over a time frame spanning the gelation time. The objective generally is to have fast enough gelation to avoid excessive flow out of the cervix and little or no flow into the fallopian tubes, while having a gelation time slow enough that the cavity uniformly fills and the catheter does not clog. These gelation times do not necessarily correspond with full crosslinking which can occur over a longer time period, but the gelation times correspond with reaching a point of crosslinking in which the hydrogel no longer is flowable, which can be evaluated in vitro under controlled conditions. Crosslinking times can depend on a combination of several factors, including relative concentrations of reactive precursors, the molar ratios of the reactive moieties, temperature, and resulting pH after mixing. Gel times may be varied by one or more of altering the pH, temperature, or buffer salt strength of the “activator” portion of the in situ system, if present.

To avoid undesirable spreading of the hydrogel, it is advantageous for the crosslinking to occur quickly. By designing the crosslinking to occur quickly, the hydrogel is less likely to migrate to off target tissues which could be damaging to off-target tissue. The crosslinking time should still allow for good flow of the hydrogel to provide cavity filling within the uterus and not clog the delivery catheter. The delivery procedure can also be designed to limit the undesirable spread of the hydrogel loaded with alcohol. As seen in the Examples, the alcohol shortens the gel times relative to comparable hydrogels without the alcohol, which can be exploited to achieve fast gel times. While a very rapid gel time may be less significant for some embodiments, generally, the gel time can be designed to be no more than about 20 seconds, in other embodiments from about 1 second (s) to about 15 s, in further embodiments from about 1.25 s to about 12 s, in other embodiments from about 1.35 s to about 9 s, and in additional embodiments from about 1.5 s to about 6 s. A person of ordinary skill in the art will recognize that additional ranges of gel time within the explicit ranges above are contemplated and are within the present disclosure. The gel times are evaluated in vitro by combining the solutions and observing the formation of precipitates. The first visual observation of precipitates is used as the gel time.

A hydrogel may be biodegradable, so that it does not have to be retrieved from the body. Biodegradability, as used herein, refers to the predictable disintegration of the material into molecules or particles small enough to be metabolized or excreted under normal physiological conditions. Biodegradability may occur by, e.g., hydrolysis, enzymatic action, cell-mediated destruction, combinations thereof, or the like. The exemplified hydrogels are believed to primarily undergo in situ degradation by hydrolysis, which is not reliant on enzymatic activity.

Hydrogels can swell upon delivery into an environment with contact with bodily fluids. While it can be desirable for the hydrogel to fill a uterine space, this can be achieved through controlling the amount of hydrogel and may not rely on swelling or exclusively on swelling. For example, the hydrogels can be adhesive to tissues. As demonstrated in FIG. 24, the hydrogels can be particularly adhesive to ablated tissue. This feature can contribute to improved diffusion of the ablation composition into the target tissue, retention of the hydrogel, and avoidance of adhesions, for example. Since the hydrogels are loaded with an ablation composition, such as alcohol, a large amount of swelling with aqueous fluids may not be desirable since it can result in dilution of the ablation composition, although if swelling is not rapid or excessive, it may not be especially significant. In some embodiments, the hydrogel can experience syneresis, or a shrinking on a weight basis, generally also on a volume basis, following initial formation relative to the initial delivery weight/volume due to a net outflow of initially trapped liquid into the surroundings, which for convenience is referred to as a negative swelling. Thus, overall swelling can be from about −25 wt % to about 200 wt %, in further embodiments from about 0 wt % to 175 wt %, in some embodiments form about 15% to about 150%, and in other embodiments from about 25 wt % to about 100 wt %. The limits of the swelling ranges can be interchanged with limits of the other ranges, such as forming a range from −25 wt % to about 100 wt %. Swelling (positive or negative) can be determined by the weight of polymer with aqueous solution of buffered saline absorbed into the polymer after 24 hours of contact with the aqueous environment relative to the weight of the polymer and absorbed aqueous solution following crosslinking into an insoluble mass, which generally occurs after several seconds. A person of ordinary skill in the art will recognize that additional ranges of swell amounts within the explicit ranges above are contemplated and are within the present disclosure.

Regardless of the crosslinking mechanism, the hydrogel can be biodegradable so that the uterine space or alternative location clears after a suitable period of time that the treatment process does not trap the hydrogel material itself. In some embodiments, the hydrogel is fully biodegraded or expelled from the body in from about 0.5 days to about 29 days, in further embodiments from about 1 day to about 21 days, in further embodiments from about 0.5 days to about 45 days, and in additional embodiments from about 3 days to about 14 days. The upper and lower ranges can be interchanged for forming alternative ranges of times, as desired. For certain applications, such as drug delivery along with ablation, it may be desirable for the hydrogel to biodegrade over a longer time period, for example, 30 days, 45 days, or longer, if the drug is designed to elute more slowly than the ablation composition. In other embodiments, it may be desirable to clear the hydrogel more quickly, such as from about 24 hours to about 72 hours. In practice, especially for intrauterine application, hydrogel is generally cleared from the body by expulsion once the hydrogel is degraded sufficiently that the modulus drops, but times can be referenced to an in situ degradation test for visible disappearance of the hydrogel in physiological solution, which is independent of patient variability. For intrauterine applications, the hydrogel is generally expunged from the uterus at least once the hydrogel loses shape, which is indicative of a significant drop in modulus due to degradation, if not sooner. In other applications, the hydrogel may persist longer prior to removal from the body. For other locations of placement within the body, the hydrogel may persist longer, and for intrauterine applications, there can be variability with respect to expulsion times. An in vitro test can examine loss of shape and disappearance of the hydrogel to provide estimates for ranges expected in vivo.

Also, the hydrogel can be selected to be soft so as to be gentle on the tissue, yet not so soft as to be extrudable from the uterus or other delivery location, resulting in unpredictable persistence within the cavity or other location. For appropriate embodiments, the intrauterine persistence of an installed hydrogel is expected to correlate with the duration of time in which the hydrogel has a modulus above a threshold range, which is estimated to be about 1 kPa to about 10 kPa. The expulsion forces would tend to be patient dependent, so that the time dependent decay of the modulus can be selected to yield values suitable to maintain the hydrogel in the uterine cavity for a majority of patients. Below this estimated range, the hydrogel is expected to have insufficient resistance to compressive forces to resist being expunged from the uterus. Specifically, the hydrogel can have an initial Young's (clastic) modulus from about 5 kPa to about 300 kPa, in further embodiments from about 8 kPa to about 250 kPa and in additional embodiments from about 10 kPa to about 200 kPa. The modulus is a measure of compressive force per unit arca divided by the change in volume per unit volume. A gel plug can be used to make the measurement using an Instron or alternative brand compressive instrument, in which the stress/strain slope is measured over the first 20% of compression. A person of ordinary skill in the art will recognize that additional ranges of degradation times and Young's modulus within the explicit ranges above are contemplated and are within the present disclosure.

With respect to alcohol delivery for ablation effect, the hydrogels for this application should have precursors and core compositions that are both hydrophilic and soluble (in uncrosslinked forms) in relatively high aqueous alcohol concentrations. As exemplified below and noted above, polyethylene glycol based hydrogel cores are suitable, although other polyethers, such as copolymers of ethylene glycol and propylene glycol, can be suitable. For example, polyvinyl pyrrolidone cores are soluble in alcohol and water, and could provide suitable hydrogel cores. The hydrogel cores are generally appropriately branched, such as by including a star polymer core, a dendrimer, a comb polymer or the like. Generally, functional groups for crosslinking are along the arms, such as terminal groups on the arms. A range of polyethylene glycol precursor macromers with medical grade are commercially available or can be custom formulated by commercial suppliers.

Reactive precursor species may have electrophilic functional groups that are, for example, carbodiimidazole, sulfonyl chloride, chlorocarbonates, n-hydroxysuccinimidyl ester, succinimidyl ester, n-hydroxyl sulphosuccinimidyl esters, sulfosuccinimidyl esters, or mixtures thereof. In some embodiments of particular interest, the electrophylic functional groups comprise N-hydroxy succinimidyl (NHS) esters, such as succinate (SS) esters or gluconate (SG) esters, that provide desirable crosslinking rates to form the hydrogel and degradation rates for the hydrogel subsequently in vivo.formed hydrogel. N-hydroxy succinimidyl succinate and N-hydroxy succinimydl gluconate are exemplified. The term synthetic means a molecule that is not found in nature, e.g., polyethylene glycol. The nucleophilic functional groups may be, for example, amine, such as primary amines, hydroxyl, carboxyl, and thiol. Primary amines can be desirable reactants with NHS electrophilic groups and provide desirable pot life stability, gel times and pH gating. The polymers in embodiments of particular interest have a polyalkylene glycol portion and can be polyethylene glycol based. The polyethylene glycol based polymer precursors can have a branched core to provide a selected number of arms that provide a plurality of crosslinking functional groups. The polymers generally also have a hydrolytically biodegradable portion or linkage, for example an ester, carbonate, or an enzymatically degradable amide linkage. The exemplified hydrogels have hydrolytically degradable ester groups. In some embodiments, a reactive precursor species can have two to ten nucleophilic functional groups each, and corresponding reactive precursor species can have two to ten electrophilic functional groups each, although as noted above, the monomers can have various numbers of arms.

The user may use visualization agents to see the hydrogel with the human eye or with the aid of an imaging device that detects visually observable visualization agents, e.g., a videocamera. A visually observable visualization agent is an agent that has a color detectable by a human eye. Suitable visualization agents include, for example, FDA approved dyes that are biocompatible. A characteristic of providing imaging to an X-ray or MRI machine is not a characteristic sufficient to establish function as a visually observable visualization agent. An alternative embodiment is a visualization agent that may not normally be seem by the human eye but is detectable at a different wavelength, e.g., the infrared or ultraviolet, when used in combination with a suitable imaging device, e.g., an appropriately equipped video camera.

The three-dimensional hydrogel structure can be resistant to expulsion from the uterine cavity, thus serving to keep the uterine walls apart and having good contact with the uterine walls.

A side benefit of the hydrogel used for ablation may be to prevent the formation of scar bridges, or adhesions. Over time the hydrogels degrade and naturally exit the uterine cavity by either systemic absorption, or mostly as discharge through the cervix and vagina following sufficient hydrogel degradation to lower the modulus accordingly.

When delivering hydrogels specifically for adhesion prevention, the hydrogels are benign, so the hydrogel can be infused until discharge is observed from the cervix. The visualization agent can be effective to facilitate this process. With the alcohol infused hydrogels or other ablation composition infused hydrogels, this may not be desirable since the ablation effect may extend then to healthy tissue that is desired to remain undamaged. The visualization agent can be useful in assessing any off target spillage of the alcohol loaded hydrogel, so it may be particularly desirable for this application. Also, it may be desirable to distend the uterus slightly through application of a slight pressure during hydrogel infusion. To achieve this, the cervix can be blocked, and hydrogel can be infused until achieving a desired delivered volume or pressure of the hydrogel in the uterus. Volume estimates can be achieved using any reasonable approach such as measuring the depth of the cavity and/or instilling and removing a sterile fluid prior to hydrogel delivery, which could also serve the purpose of flushing the cavity. In some embodiments, a rough estimate of the infusion volume can be used to corroborate sensing of pressure through appropriate means. The catheter manifold used to connect the syringe to the catheter can comprise an extra Y-branch to which a pressure sensor can be attached. Commercial pressure sensor components for use as a pressure sensor component of the catheter fittings are commercially available, for example, from PendoTECH, Princeton, NJ, USA.

Hydrogel applicators have been developed for intrauterine delivery of hydrogel precursors. Syringe-based applications for the simultaneous delivery of two solutions for hydrogel in situ/in vivo formation have been generally developed and have been used with commercial medical hydrogel products. Adaptation of these applicators for intrauterine delivery is described in the '086 application cited above. Significant improvements to the device design for intrauterine application are found in the '276 application. The devices described in the '276 application are particularly useful for hydrogel delivery when the cervix is dilated but can also be readily used when the cervix is not dilated. In the present context, the cervix is generally not necessarily dilated, although in particular cases, it may be partially dilated.

For adhesion prevention as described in the '276 application, some discharge of hydrogel precursor can be used to verify that sufficient amount of hydrogel has been delivered, while the device then provides for blocking further discharge to maintain the hydrogel in place. For the present embodiments, it is more significant to maintain the hydrogel within the uterine cavity while nevertheless appropriately filling the uterine cavity, and possibly with some positive pressure to push out the uterine lining to better achieve good surface contact. Also, it may be desirable to flush out the uterine cavity to remove any blood or other matter that could interfere with the ablation. Also, fluid can be removed to avoid dilution of the hydrogel and the alcohol content. The applicator from '276 application can provide for flushing and/or aspirating fluids from the uterine cavity. In general, syringes or the like can be used to provide desired fluid exchanges.

As noted above, dual syringe applicators can be adapted for the delivery precursors of the alcohol (or other ablation composition) loaded hydrogels with appropriate solutions loaded into the respective syringes. Since the volumes of the solutions may not be equal, different syringe volumes can be used for the respective syringes. The syringe plungers can be simultaneously depressed to deliver the solutions form the syringes into a manifold where the solutions are mixed and delivered from the manifold into an appropriate catheter. The catheter can be selected to be appropriate for the particular application. For example, for delivery into a tumor, wart, or precancerous tissue lesion, the catheter can be a needle of an appropriate gauge for delivering the desired volume in an appropriate time to avoid clogging the syringe. As used herein, catheter refers to soft, flexible catheters, needles, or cannula, which are suitable for medical applications. In the following, specific applicators are described for intrauterine delivery to fill the uterine cavity or for delivery to a fallopian tube. Other applications can be used for additional applications as desired.

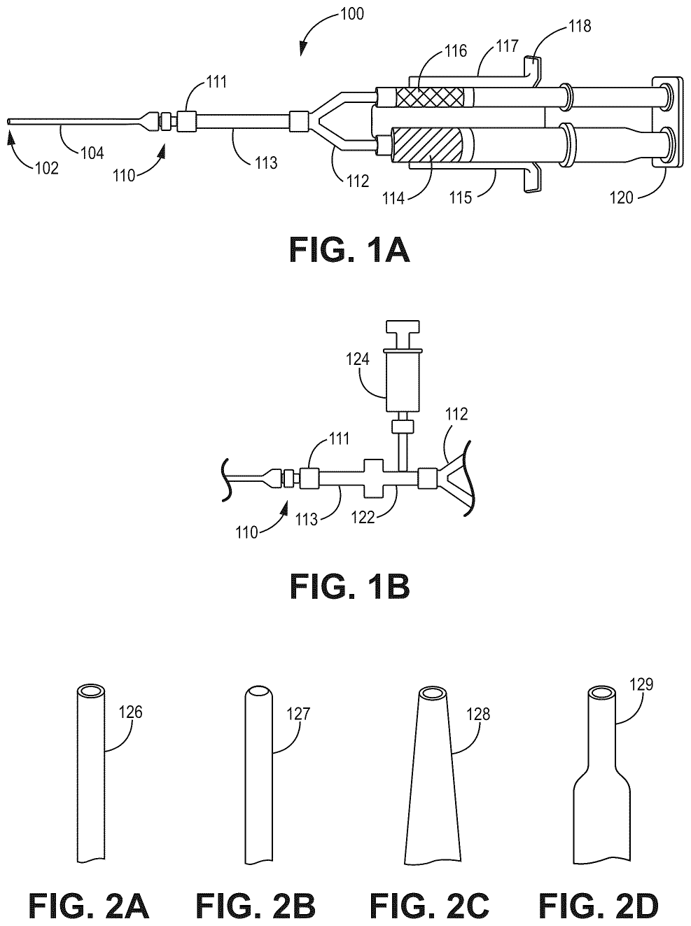

FIG. 1A displays an applicator suitable for a two-component hydrogel precursor system. If more than two components are delivered, such as three, four or more components, the figures can be generalized by a person of ordinary skill in the art based on these teachings. Similarly, other fluids, such as flushing fluids, can be delivered similarly to the hydrogel precursor system. Also, with an empty syringe compartment, the catheter system can be used to extract out fluid, which can be any composition that can be pulled into the catheter.

### Free Radical Crosslinked Hydrogels

The use of free radical polymerized hydrogels provides a suitable system for the delivery of ablation compositions, especially for ablation compositions that are not compatible with pH gated hydrogel polymerization. Thus, free radical polymerization can be used in the presence of acidic compositions, such as acetic acid, trichloroacetic acid or the like. For these crosslinking approaches, a single precursor type can be used, in which the functional group is a free radical polymerizable group. Free radical polymerization generally involves an initiator that starts a chain reaction that can eventually terminate by one or more mechanisms. The amount of radical initiators can be provided to provide suitable completion of crosslinking. Initiators can be triggered by radiation or chemical reactions. The focus herein is on use of redox (reduction-oxidation) oxidation reactions to trigger the radical addition reactions, but radiation can be similarly used if desired.

Suitable functional groups for free radical crosslinking generally include a vinyl group as discussed above. The hydrophilic cores are similarly discussed above, and polyethylene glycol cores rely on established technology for medical applications. Acrylic acid species and derivatives thereof provide a vinyl group for crosslinking and a carboxylate group for attachment to a hydrophilic polymer core. Branched PEG acetate precursors, as well as PEG precursors for nucleophilic electrophilic crosslinking, are available form Jenkem USA, Plano, TX. Suitable initiators and co-initiators for activating free radical polymerization are also discussed above. During delivery, the constituents are blended and begin crosslinking with gelation upon delivery into the target treatment location. The crosslinking reactions terminate upon appropriate completion of the crosslinking, which may be formation of a fully crosslinked material.

Injection into a space such as the intrauterine cavity results in the free radical polymerized hydrogel forming a space filling mass if the precursor solution has sufficient solid content. Since the crosslinking is not pH gated, the ablation composition can be acidic without hindering the crosslinking. Acid can result in degradation of the crosslinked polymer. The amount and concentration of acid can be adjusted to provide a desired degree of ablation prior to excess degradation of the hydrogel. The crosslink density can be adjusted to balance elasticity of the crosslinked hydrogel and the degradation time in situ. Acetic acid is less acidic than halo-acetic acids, so acetic acid may provide a better balance of desired efficacy for ablation and reduced effect on polymer matrix materials.

Other features of free radical polymerized hydrogels can adopt similar features as the electrophile-nucleophile crosslinked hydrogels. For example, the crosslinking density to can be selected to achieve a comparable modulus for the gelled material. The properly selected modulus can aid with retention of the loaded hydrogel at the treatment site, such as the intrauterine cavity. Also, visualization agents can be similarly incorporated into the hydrogel at appropriate concentrations for visualization. The system for forming the radical crosslinked hydrogel can be similarly delivered using a comparable, dual-syringe applicator, and it may be desirable to deliver a lower volume of activator solution with an initiator or co-initiator relative to the volume of precursor with ablation composition. The applicator can combine the precursor solution and the activator solution for flow through a catheter to the delivery site.

### Microparticles

The delivery of ablation compositions can be controlled with the optional use of microparticles. The microparticles can sequester the ablation compositions, or a portion thereof, for gradual release from the microparticles as well as the hydrogel. Generally, the microparticles are embedded within the hydrogel following gelling in situ. The microparticles are generally delivered with the precursors such that gelling of the hydrogel traps the microparticles similarly to blended ablation compositions. The ablation compositions can be initially fully sequestered in the microparticles or divided between the microparticles and the hydrogel free of the microparticles. The ablation compositions can escape from the microparticles due to degradation of the microparticles and/or diffusion from the microparticles. The rate of delivery to adjacent tissue depends on availability of the ablation composition to reach the tissue. The microparticles engorged with the ablation composition can be formed by absorbing the ablation composition into the microparticles during formation of the microparticles and/or after forming the microparticles. The degradation behavior can be selected to be suitable for the desired degree of ablation, the nature of the ablation compositions, and the various concentrations of species. As used herein, the term microparticles refers to microscopic distinct phases embedded in a hydrogel regardless of the specific morphology, shape or other characteristics, unless specifically noted otherwise.

The use of microparticles or the like embedded in hydrogels was developed to facilitate and control delivery of therapeutics for in vivo application. With hydrogels formed in situ, this is described in published U.S. 2016/0166504 to Jarrett et al. (hereinafter the '504 application), entitled “Hydrogel Drug Delivery Implants,” and published U.S. patent application 2018/0085307 to Sawhney et al., entitled “Intracameral Drug Delivery Depots,” both of which are incorporated herein by reference. Suitable biodegradable polymers for forming microparticles include, for example, polymers and copolymers of: poly(anhydride), poly(hydroxy acid)s, poly(lactone)s, poly(trimethylene carbonate), poly(glycolic acid), poly(lactic acid), poly(lactic acid)-co-poly(glycolic acid) (PLGA), poly(orthocarbonate), poly(caprolactone), crosslinked biodegradable hydrogel networks like fibrin glue or fibrin sealant as well as polyethylene glycol and other polymers suitable for the in situ formed hydrogel matrix, or mixtures thereof. PLGA is known to have a hydrolysis rate that depends on the ratio of the monomers. The polymers of the microparticles may be degradable by hydrolysis and/or enzymatic action. For embodiments with microparticles, the polymers can be selected to be compatible with the ablation composition.

In some embodiments, the microparticles can be formed by milling a polymer to form particles of desired sizes. The milling can be performed using mechanical mills, such as ball mills, mortar and pestle, blenders, homogenizers, or the like, and various brands and models of these instruments are commercially available. The milled particles can be sieved or otherwise sorted if it is desired to have a narrower particle size.

For use herein, the microparticles generally have an average diameter of no more than about 250 microns, in further embodiments from about 0.25 microns to about 150 microns and in other embodiments from about 0.5 microns to about 100 microns. A diameter can be considered as an average of the longest dimension along a principal axis and the shortest dimension along a principle axis. A person of ordinary skill in the art will immediately appreciate that all the ranges and values within the explicitly stated ranges are contemplated, e.g., from about 0.3 to about 10 μm or from about 1 to about 30 μm and are within the present disclosure. A simple method for sizing particles involves using custom-made or standardized sieve mesh sizes. Another method to measure particle size is with a laser diffraction particle size analyzer, such as the Coulter LS 200, which analyzes particles when suspended in a liquid such as saline. Light scattering measurements can be performed on a sample, which may be considered a destructive test. The term particle is intended to include structures of any shape and irregularly shaped particles. A spheroidal particle refers to a particle wherein the longest central axis (a straight line passing through the particle's geometric center) is no more than about twice the length of other central axes. A rod-shaped particle refers to a particle with a longitudinal central axis more than about twice the length of the shortest central axis. Suitable collections of particles can include a blend of a plurality of separately characterized collections of particles, with the separately characterized collections potentially having different average particle sizes, different compositions, different rates of degradation in vivo, and potentially other distinct properties.

The microparticles can be formed through polymerization or crosslinking in the presence of the ablation composition to form loaded microparticles. In other embodiments, the loaded microparticles can be formed through a phase separation. For example, a polymer, such as PLGA, which is insoluble in water, can be mixed with an ablation composition, such as acetic acid, and then water can be added to form microspheres directly or with further processing, where the acetic acid partitions between the microparticles and the water. The amounts of the species can be adjusted to provide the desired properties following phase separation. The phase separated particles can be filtered off and further processed. In still further embodiments, the microparticles can be incubated in a solution of the ablation composition to absorb the ablation composition into the microparticles. The contact time for the absorption whether or not including the time for forming the microparticles can be in some embodiment at least about 10 minutes and in further embodiments from about 15 minutes to about 48 hours. Unless the microparticles degrade in the ablation solution, longer absorption times can be used without detriment.

Microparticles loaded with an ablation composition can also be formed using electrospraying. This approach provides other potential processing options. See, for example, Wang et al., “Electrospraying: Possibilities and Challenges of Engineering Carriers for Biomedical Applications—A Mini-Review,” Frontiers in Chemistry 7, April 2020, incorporated herein by reference.

The microparticles loaded with an ablation composition can be mixed with the hydrogel precursors prior to delivery into the patient. Depending on the desired dose of the ablation composition, the microparticle loading in the hydrogel precursor may be relatively high. In some embodiments, the precursor solution can comprise from about 5 weight percent to about 75 weight percent microparticles, in further embodiments, from about 10 weight percent to about 70 weight percent, and in other embodiments from about 15 weight percent to about 65 weight percent microparticles. A person of ordinary skill in the art will recognize that additional ranges of microparticle content within the explicit ranges above are contemplated and are within the present disclosure. The microparticles can comprise a weight percent of ablation composition, which can be a liquid. The solid content of the microparticles would be correspondingly based then on the solid polymer. The hydrogel precursors can be dissolved in water, alcohol-water blends, saline or other reasonable biocompatible solvent or solvent blends in which the precursors are soluble and the microparticles can be dispersed.

The overall solids content of the hydrogel precursor solutions can be determined based on the amount of the amount of hydrogel precursors and the microparticle polymers. The precursor solution loaded with microparticles delivered from the applicator has a solid content from about 3 weight percent (wt %) to about 25 wt %, in further embodiments from about 3.5 wt % to about 20 wt %, and in other embodiments from about 3.75 wt % to about 17.5 wt %. A person of ordinary skill in the art will recognize that additional ranges of solid content within the explicit ranges above are contemplated and are within the present disclosure.

### Endometrial Ablation

Referring to FIG. 1A, an illustrative transcervical access system constructed in accordance with the principles of the protocols herein is described. Transcervical access system 100 comprises single-lumen catheter 104 having proximal end 110 and distal end 102. Distal end 102 generally comprises one or more delivery ports, as described in detail below. In some embodiments, transcervical access system 100 has an egress limiter along catheter 104. As described below, an egress limiter can be used to set a depth for insertion of the catheter into the patient.

Catheter 104 connects to a Y-connector 112 at fitting 111, such as a luer fitting or the like. Y-connector 112 can comprise a static mixing element, such as within tubing segment 113. Tubing segment 113 generally has a larger diameter than catheter 104 and is stiffer. Tubing segment 113 can provide for convenient manipulation of catheter 104 for insertion into the patient and provide the practitioner better positioning for the procedure while limiting the length of catheter 104, which may be more difficult to manipulate if it is too long. In any case, mixing of first solution 114 and second solution 116 occurs in or begins within Y connector 112. Y-connector 112 can comprise the mixing element in its outflow channel, or a separate section of tubing connected to the Y-structure, and the static mixing element can comprise flow-altering baffles, such as a helical, plate-type or other flow diverter known in the art to induce turbulence within a tubular channel, to encourage appropriate mixing of the solutions. Y-connector 112 has a fitting 111, such as a Luer connector for attachment of catheter 104, and connections to syringes 115, 117, which may or may not be releasable. First solution 114 can be a mixture of a first precursor and a second precursor and the second solution can be an activator/catalyst solution. Syringes 115 and 117 generally are held via molded syringe holder 118 or the like to provide for convenient handling by the health care professional during use. An optional fixed ratio of solution delivery ratio can be maintained by an optional plunger cap 120. If the internal diameters of syringes 115, 117 are the same, movement of plunger cap 120 would deliver a 1:1 ratio of volumes, but the internal diameters can be selected to provide a different volume ratio if desired. The outer diameters of syringes 115, 117 may or may not track the inner diameters depending on the syringe wall thicknesses. FIG. 1A is depicted with syringe 115 having a larger internal diameter than syringe 117. As noted herein, the precursor solutions can be desirable delivered as a ratio different from 1:1 to provide a desired alcohol concentration.

An alternative embodiment of transcervical access system 100 is depicted in FIG. 1B. In this embodiment, T-branch fitting 122 is located within Y-connector 112 proximal to tubing segment 113. In some embodiments, T-branch fitting 122 can be secured with connectors, such as Luer connectors on the respective ends of the three branches, although in some embodiments, T-branch fitting 122 can be molded to be integral with one or more of adjacent components. Similarly, another Y-branch or other fitting design can be used in place of T-branch fitting 122. T-branch fitting 122 can be connected to syringe 124 or other fluid source, which can provide an inert flush, such as buffered saline, to help clear the catheter or for the delivery of a therapeutic or other desirable liquid. Also, a negative pressure source, such as an empty syringe with the plunger withdrawn at least partially or a pump, attached to T-branch fitting 122 can be used to aspirate fluid through the catheter.

FIGS. 2A-2D are fragmentary views of optional embodiments of basic tips for distal end 102. Tip 126 has an open end to a cylindrical catheter. Tip 127 has a tapered open end to a cylindrical catheter. Tip 127 can provide smoother placement of distal end 102 into narrow openings. Tip 128 has an open end that connects to a cylindrical catheter via a continuous taper. In some embodiments, Tip 128 is a terminal segment of a continuously tapered catheter. Tip 129 has an open end that steps-down in diameter to connect to a cylindrical catheter. Tips 127-129 may provide smoother placement of distal end 102 into narrow openings. Results shown in the Examples below demonstrate that with appropriate hydrogel flow properties, installation tip 126 can provide desirable filing of the uterus without clogging of the tip. In alternative embodiments, openings from the catheter can direct the hydrogel radially away from the catheter to fill the uterus volume, and for these embodiments, the distal end may be closed.

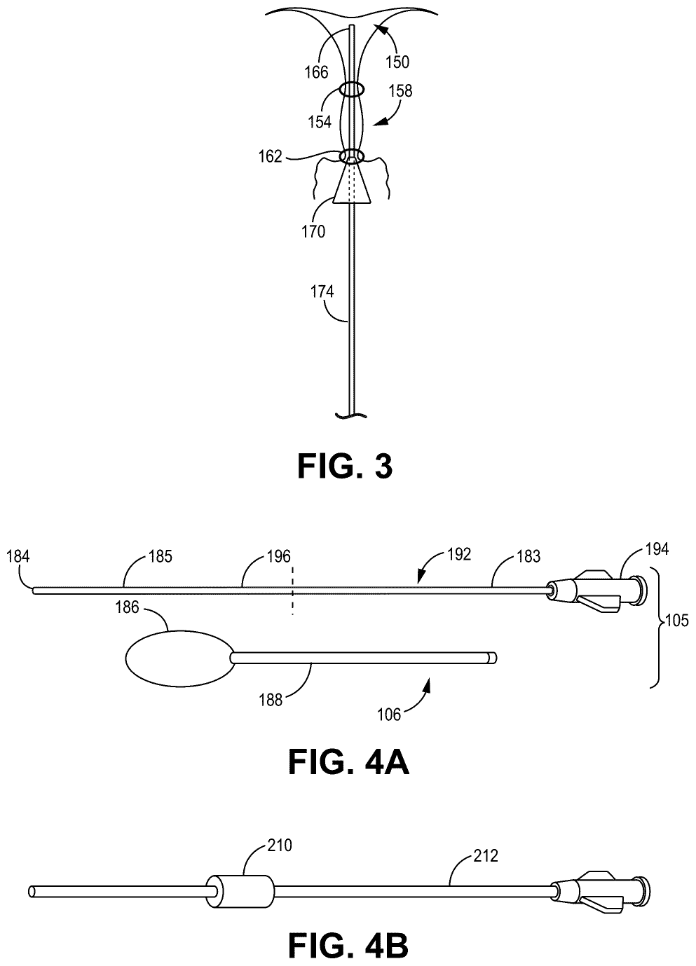

FIG. 3 shows uterine cavity 150, installation tip 166, internal os 154, endocervical canal 158, which is approximately 4 cm in length, external os 162, cap element 170, and catheter 174. Generally, for a particular patient, the health care provider knows with reasonable accuracy the patient's uterine anatomy relating to the uterine length and the length of the endocervical canal such that a closure on the catheter can be adjusted to provide the prescribed distance of the catheter tip from the back of the uterus. The distance from the catheter tip to the back of the uterus following placement of the catheter can be from about 0.25 cm to about 2.0 cm and in further embodiments form about 0.35 to about 1.25 cm. A uterine sound instrument can be used to evaluate the distance using conventional procedures. If an egress limiter is used, the sound can be held next to the egress limiter to adjust the position of the cap. A person of ordinary skill in the art will recognize that additional distance ranges within the explicit ranges above are contemplated and are within the present disclosure.

FIG. 4A shows an embodiment in which catheter assembly 105 is shown with separated catheter 192 and egress limiter 106. Egress limiter 106 comprises cap element 186 and tubular member 188 proximal to cap element 186. Cap element 186 can take various shapes as desired. With an undilated or only slightly dilated cervix, a cup shaped cap element can be suitable with the open edge of the cap facing toward the cervix. Tubular member 188 is supported by the catheter during infusion and is generally less flexible than the length of catheter 192 distal to cap element 186. Catheter 192 comprises a connector or hub 194 and a tubular element 196. Length of catheter 192 is designed for insertion into the uterine cavity with cap element 186 against the outer opening into the cervix, referred to as the external orifice or external os. Tubular member 188 can have an adjustable position when assembled to correspond with overlapping all or a portion of the length of tubular element 196 from cap element 186 to connector or hub 194. Furthermore, tubular element 196 may or may not be uniform along its length with respect to structure and/or composition. As noted above, the catheter tip can be very soft to avoid tissue injury during hydrogel infusion, but a very soft polymer can make manipulation of the applicator more difficult if incorporated along the entire length of tubular element 196. In some embodiments, tubular element 196 comprises a distal port 184 and in some embodiments includes a tip, and a proximal portion 183 that is stiffer than distal portion 185, which is shown with a dashed line to divide these regions. Optional positions for separating a stiffer proximal region is described further below. Proximal portion 183 can be formed from a section of tubing secured over the catheter, a change in material of the catheter, and/or a thickening of the catheter wall. The embodiment with a stiffer proximal portion 183 depicted in FIG. 4A gives the user greater stability when instrumenting the endocervical canal.

Referring to FIG. 4A, the proximal end of cap element 186 and tubular member 188 to provide egress limiter 106 are shown in a separated configuration. In an assembled configuration, tubular member 188 provides external stiffening to at least a part of the length of catheter 192 proximal to the cap element. Tubular member 188 also allows for gripping egress limiter 106 to facilitate the procedure. Tubular member 188 can have a length from about 5 cm to about 20 cm, in further embodiments from about 6 cm to about 19 cm, and in some embodiments from about 7 cm to about 18 cm. A person of ordinary skill in the art will recognize that additional ranges of length within the explicit ranges above are contemplated and are within the present disclosure. When assembled, egress limiter 106 is engaged over part of catheter 192 generally for insertion of the catheter into the patient. Egress limiter 106 allows the user to adjust the position of cap element 186 to give values of the distal catheter length in the ranges as specified above. In some embodiments, tubular member 188 can internally provide a friction interaction with the catheter surface to restrict unintended motion of the position of egress limiter 106. The user can set the position of egress limiter 106 and maintain the position along catheter 192 by avoiding unintended sliding of tubular member 188. In other embodiments, the cap element 186 position can be adjusted and/or maintained via a clip, ridges that engage a flange on the mated element, or the like between catheter 192 and egress limiter 106. Since the cap element 186 is fixedly attached to tubular member 188, the design of tubular member 188 prevents the inadvertent loss of the cap element 186.

In some embodiments, it can be desirable to deliver a plug into the cervix to assist in controlling the stabilization of the delivered hydrogel into the uterus and the inner OS of the cervix. Referring to FIG. 4B, a plug 210 can have an inner lumen providing for it to slide over catheter 212. Plug 210 can be placed into the cervix prior to delivery of hydrogel precursors into the uterus, and plug 210 can be left in place following removal of the catheter. Plug 210 may swell in contact with moist tissue such that expansion of the cervical plug can help to maintain the position of the cervical plug when the catheter is removed. Alternatively or additionally, the cervical plug can be held in place by the health care professional when the catheter is removed. In some embodiments, plug 210 protects the cervix from unintentional contact with the hydrogel precursor system and/or the alcohol-loaded hydrogel. For use with an un-dilated cervix, a cervical plug may have a thickness on the order of a millimeter.

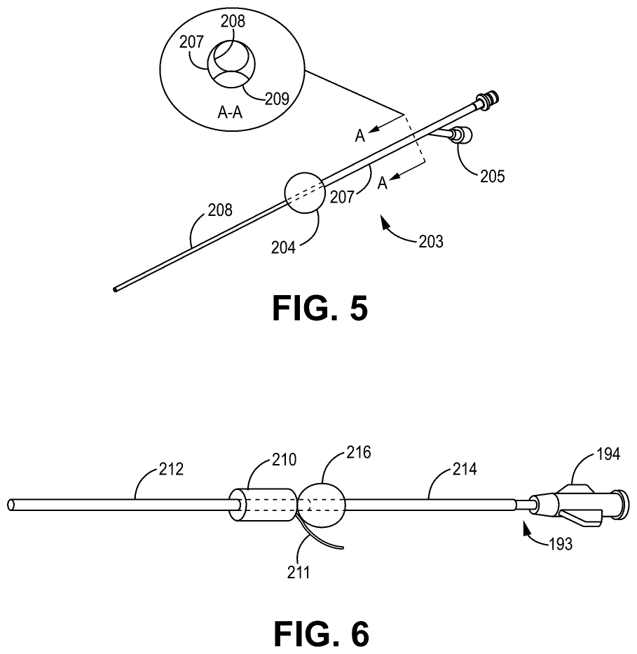

FIG. 5 illustrates an embodiment in which catheter assembly 203 has an egress limiter comprising a cap element 204 that is inflatable, like a balloon, and can be filled with gas or fluid through port 205 to customize the size for the desired fit within the cervix. In some embodiments the fluid can be air, saline or other fluid. An uninflated balloon can have a very low profile along the catheter body. Port 205 can have a terminal end that has a luer connector or the like for attachment with a device to deliver and/or remove fluid. In some embodiments, a syringe or the like can be used to inflate cap element 204 through port 205 to achieve a desired volume, such as through a luer connector. Generally, port 205 connects with a balloon lumen to cap element 204. For example, as shown in a cross-section in the inset of FIG. 5, tubular member 207 may have a balloon lumen 209 while providing for a slidable engagement over catheter 208. In general, a balloon lumen can have any configuration known in the art, such as concentric or non-concentric as shown in the insert of FIG. 5. In some embodiments, after the installation of the hydrogel, cap element 204 may be deflated through port 205 to facilitate easier removal of tubular member 207. Tubular member 207 along with port 205 can be slid off catheter 208 to allow for removal of catheter 208 while holding tubular member 207 with cap element engaged with the outer OS. In some embodiments, the deflation may use a syringe or other negative pressure device.

It can be desirable to use both a cervical plug and an egress limiter to control fluid delivery. In particular, the egress limiter can facilitate proper placement of the plug and maintenance of the plug during fluid delivery. Referring to FIG. 6, catheter assembly 193 comprises catheter 212, egress limiter 214, and plug 210. Plug 210 can be placed to abut cap element 216 to anticipate appropriate placement of plug 210 when cap element is placed at the outer OS of the cervix. Plug 210 can be connected to a tether 211 to facilitate removal of plug 210 if desired, especially if the plug is not biodegradable.

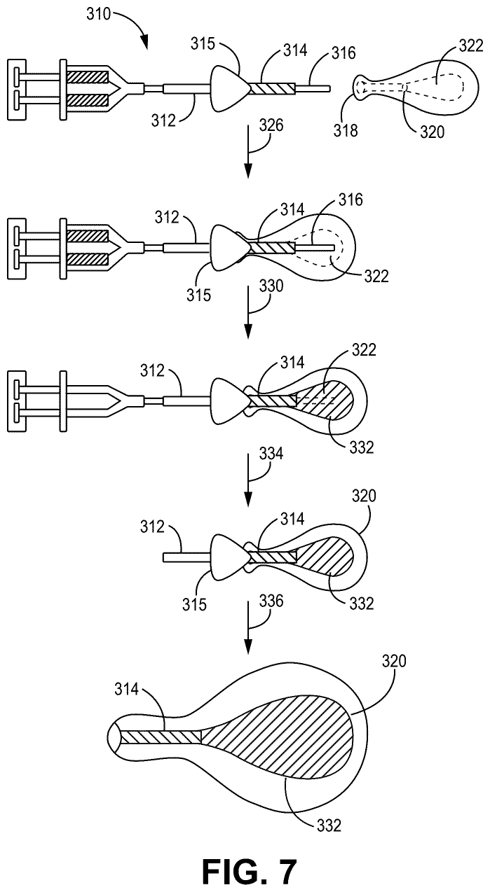

Referring to FIG. 7, the top image depicts an applicator 310 with an egress limiter 312 and a cervical plug 314 distal to the cap element 315 of egress limiter 312 mounted over catheter 316 positioned for insertion into outer os 318 of the cervix into the uterus 320 for placement of the catheter tip in the uterine cavity 322. Following insertion 326, the second image of FIG. 7 shows the tip of catheter 316 in uterine cavity 322 with cervical plug 314 in the cervix and cap element 315 positioned at the outer os of the cervix. Following injection 330 of hydrogel precursors, the third image of FIG. 7 depicts hydrogel 332 within uterine cavity 322 up to cervical plug 314. Following removal 334 of catheter 316 from uterus 320, hydrogel 332 fills the uterus, cervical plug 314 is in place within the cervix, and egress limiter 312 is in position with cap element at the outer os of the cervix. Following removal 336 of egress limiter 312, the last image of FIG. 7 depicts uterus 320 filled with hydrogel 332 with cervical plug 314 still in place.

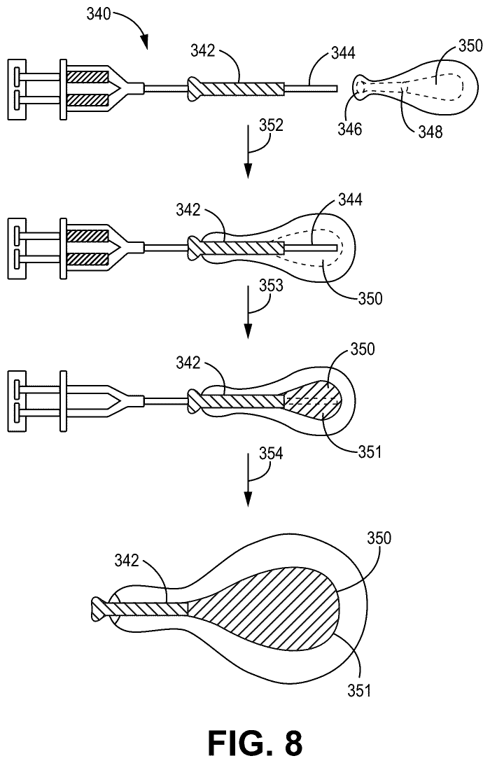

Referring to FIG. 8, the upper image depicts an applicator 340 comprising a capped cervical plug 342 on catheter 344 positioned for insertion through outer os 346 of the cervix leading into uterus 348 within uterine cavity 350. Capped cervical plug 342 combines features of a cervical plug and an egress limiter in a unitary structure. Upon insertion 352, the second image depicts the tip of catheter 344 in uterine cavity 350 with cervical plug passing through the cervix with the capped end at the outer os of the cervix. Upon the delivery 353 of hydrogel precursors shown in the third image of FIG. 8, hydrogel 351 fills uterine cavity 350. Upon removal 354 of catheter 344, the fourth image of FIG. 8 depicts hydrogel 351 filling uterine cavity 350 with capped cervical plug 342 remaining in place.

In summary, for endometrial ablation, the precursor solutions are generally mixed near the start of the procedure. Precursor powders are mixed with appropriate solvents for each tube, generally taken from sterile containers. The mixed solutions are placed into respective syringes if they are not mixed directly in the syringes, which can be done if desired. The mixing of the solutions starts the timing for the pot life for use of the solutions. Once a desired insertion length is determined, an egress limiter can be set at a desired position, if used, and the catheter can be inserted into the patient. The catheter may or may not be connected to the Y-connector prior to insertion and the syringes may or may not be connected to the Y-connector prior to connection to the catheter.

Generally, the components of the system should be desirably positioned prior to initiating any precursor infusion. Also, the desired volume can be estimated, and/or a mechanism for evaluating status of infusion can be ready for evaluating a stop point prior to initiating infusion. The infusion can then be started once everything is ready. Generally, it is undesirable to pause infusion for any significant period of time since gelling of the hydrogel can then clog the catheter. Due to the small catheter diameter, the catheter volume is very small, so even at a relatively slow infusion rate, the dwell time in the catheter can be small. Once the desired hydrogel volume is delivered, infusion can stop. If an egress limiter is used, the system is designed such that the catheter can be removed with the egress limiter in place, which inhibits pulling out hydrogel from the uterus with the catheter. Once the catheter is safely removed, the egress limiter can be correspondingly removed. A range of installation of ablation chemical-loaded hydrogel is contemplated from about 90% to about 300% of the uterine volume, generally an undistended uterus, in further embodiments from about 110% to about 200% and in some embodiments from about 120% to about 175%. A person of ordinary skill in the art will recognize that additional ranges of hydrogel installation volumes within the explicit ranges above are contemplated and are within the present disclosure.

### Fallopian Tube Ablation

Loaded hydrogels can provide desirable functionality with respect to ablating a portion of the fallopian tubes as a means of birth control or other medical reasons. The hydrogels can be effective to conform to the irregular surfaces of the fallopian tubes, and the constrained space can be consistent with constraining the alcohol to an acceptable space without blocking the distal end of the tube toward the ovaries. A specific applicator is described, although other devices can be used as desired.

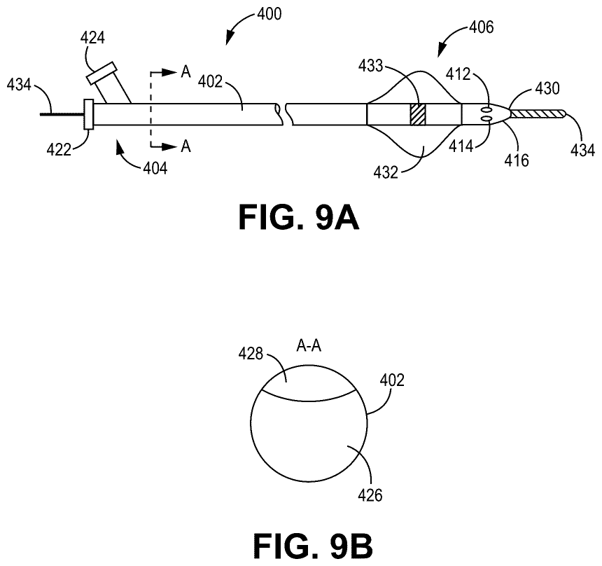

FIGS. 9A and 9B display a delivery system applicator suitable for the delivery of the alcohol-based hydrogel precursor system described herein to chemically ablate a body lumen, such as a fallopian tube. Delivery system 400 comprises multi-lumen catheter 402 having proximal end 404 and distal end 406. Proximal end 404 includes inlet port 422 which is coupled via lumen 426 to outlet port 412, 414, 430 disposed near tip 416. Balloon inflation port 424 is coupled via balloon lumen 428 to balloon 432. A dual syringe delivery system such as shown in FIG. 1A can be connected to inlet port 422 for precursor delivery. The two lumen 426, 428 can be seen in the cross-sectional view of FIG. 9B. In this embodiment, lumen 426 functions both as a guidewire lumen and a lumen for the delivery of the hydrogel precursor system. With guidewire 434 in place, outlet port 430 is substantially blocked for exit of the hydrogel precursor system. Alternatively, in operation, lumen 426 initially may be used to position delivery system 400 in a desired position, and guidewire 434 is then withdrawn. The hydrogel precursor system is then injected through inlet port 422 and into lumen 426, and finally exiting through outlet port 430, and outlet ports 412, 414 may still be used or removed.

Radio-opaque marker band 433 is disposed within balloon 432, or elsewhere on distal end 406, to assist in positioning distal end 406 of delivery system 400 within a body lumen under fluoroscopic guidance. Guidewire 434 extends through inlet port 422 and outlet port 430, and may be used, for example, to intraluminally guide tip 416 of delivery system 400 to a treatment site, such as a fallopian tube, a peripheral blood vessel, aneurysm, or other bodily lumen. Balloon 432 may be inflated to anchor catheter 402 in position within a body lumen during formation of a hydrogel implant, and may also occlude a lumen to prevent fluid flow from diluting the hydrogel precursor system during gelation. Delivery system 400 optionally may include an outer sheath that surrounds balloon 432 when the balloon is deflated.

Alternatively, catheter 402 may be configured to have three lumens, for example, a precursor formulation precursor lumen, an activator solution lumen, and a balloon lumen, thus providing separate lumens for the precursor formulation and the activator solution, the two solutions of the hydrogel precursor system. In some embodiments, the separate lumens may merge such that a mixed, activated formulation is delivered to outlet ports 412, 412, and 430. In other embodiments. In other embodiments, a precursor formulation lumen couples a precursor formulation inlet port to outlet ports 412 and 414, an activator solution lumen couples inlet port 422 to outlet port 430, and balloon lumen 428 couples balloon inflation port 424 to the interior of balloon 432. The hydrogel precursor system crosslinks after placement in the body lumen. As a further alternative to the embodiment of FIGS. 9A and 9B, the guidewire lumen need not extend the length of the catheter to form a so-called “over the wire” catheter. Instead, the guidewire lumen may be configured as a shorter lumen that exits catheter 402 through a skive just proximal of balloon 432 to form a so-called “rapid exchange” catheter, as described, for example, in U.S. Pat. No. 4,762,129 to Bonzel.

Delivery system 400 should be of a size appropriate to facilitate delivery, to have a low profile, and cause minimal trauma when inserted and advanced to a treatment site. In an embodiment suitable for forming alcohol-loaded hydrogel implants in fallopian tubes to effect chemoablation, delivery system 400 can be no larger than about 1.6 mm (0.065 inches) to allow delivery through a non-dilated or slightly dilated cervix, optionally using a standard coronary guide catheter. The device may be sized to be deliverable over small diameter guidewires, such as guidewires having a diameter of approximately 0.30-0.45 mm (0.012-0.018 inches), as commonly used in the coronary arteries.

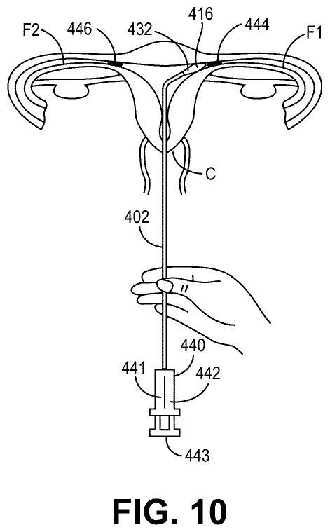

Referring to FIG. 10, a method of using delivery system 400 of FIG. 9A is described for simultaneously delivering the precursor formulation and activator solution into a fallopian tube lumen to form an alcohol-loaded hydrogel. To avoid contact of the ablating hydrogel with off-target tissue, the proximal end of the tube near the uterus can be blocked, such as with a balloon that can be inflated or deflated as appropriate, and the volume of hydrogel precursors can be sufficiently low to avoid hydrogel leaking from the distal end of the tube near the ovaries. An appropriate precursor volume can be from about 0.1 milliliter (ml) to about 2 mls. Fallopian tube F1 is accessed by passing catheter 402 through cervix C under fluoroscopic guidance. Proximal end 404 of delivery system 400 is coupled to dual syringe-type device 440 containing precursor formulation 441 and activator solution 442 and having actuator 443 that allows controllable injection of precursor formulation 441 and activator solution 442 into catheter 402. In some embodiments, dual syringe-type device 440 connects to catheter 402 via a Y-connector to simultaneously direct precursor formulation 441 and activator solution 442 into lumen 426. In additional embodiments, a mixing unit is provided between catheter 402 and the Y-connector. In other embodiments, dual syringe-type device 440 connects to a multi-lumen catheter with separate lumens for precursor formulation 441 and activator solution 442. Balloon 432 may be inflated with a fluid containing a contrast agent to verify placement of tip 416.

If desired, following inflation of balloon 432, the treatment space may be filled or flushed with a solution, such as an inert saline solution, to remove blood and other physiological fluids from the treatment space. Delivery system 400 optionally may include an additional lumen or use an existing lumen, such as lumen 426, to permit such flushing liquids to exit the treatment space. Alternatively, a non-inert solution, such as a solution containing a pharmaceutical agent, may be injected into the treatment space.

Actuator 443 is then depressed so that precursor formulation 441 and activator solution 442, mixed or separately, are delivered within the fallopian tube through one or more outlet ports distal to balloon 432. In some embodiments, the delivery time can be no more than about 1 second, no more than about 3 seconds, no more than about 5 seconds, or no more than about 20 seconds. The precursor formulation crosslinks after mixing with the activator solution thus forming alcohol-loaded hydrogel plug 444 within fallopian tube F1. Balloon 432 is then deflated and catheter 402 withdrawn. The procedure above may be repeated to form alcohol-loaded hydrogel plug 446, either with the same catheter and Y connecter if not plugged, or a new catheter and Y connecter if plugged following the first application. Plugs 444 and 446 gently chemically ablate the fallopian tube tissue that is in contact with the plug and the alcohol that diffuses from the plug. Plugs 444 and 446 degrade and leave ablated tissue that scars and/or forms adhesions that block the fallopian tube. The method provides a minimally invasive, non-surgical method of sterilization.

After a period of time to provide for healing and scarring completion, a tubal patency test can be performed to confirm effectiveness of the procedure. Such test are known in the art, such as a tubal insufflation test (Rubin test) involving the blowing of air, or sonosalpingography involving the delivery of saline under ultrasound imaging. The period of time is not generally important, but usually at least two weeks after clearing the hydrogel would be appropriate.

### Other Applications

Procedures for other applications can be adapted from the teachings herein using appropriately modified applicators. For example, for tumor applications, an appropriate needle gauge can be used to inject the hydrogel, and slightly longer gel time can allow the precursors to profuse through more tumor tissue prior to gelling. The procedures can be repeated at appropriate time intervals if needed.

### EXAMPLES

### Example 1: Effect of Alcohol Concentration on In Vitro Performance

This example demonstrates the effect of alcohol concentration and pot life on the performance of precursor formulations.

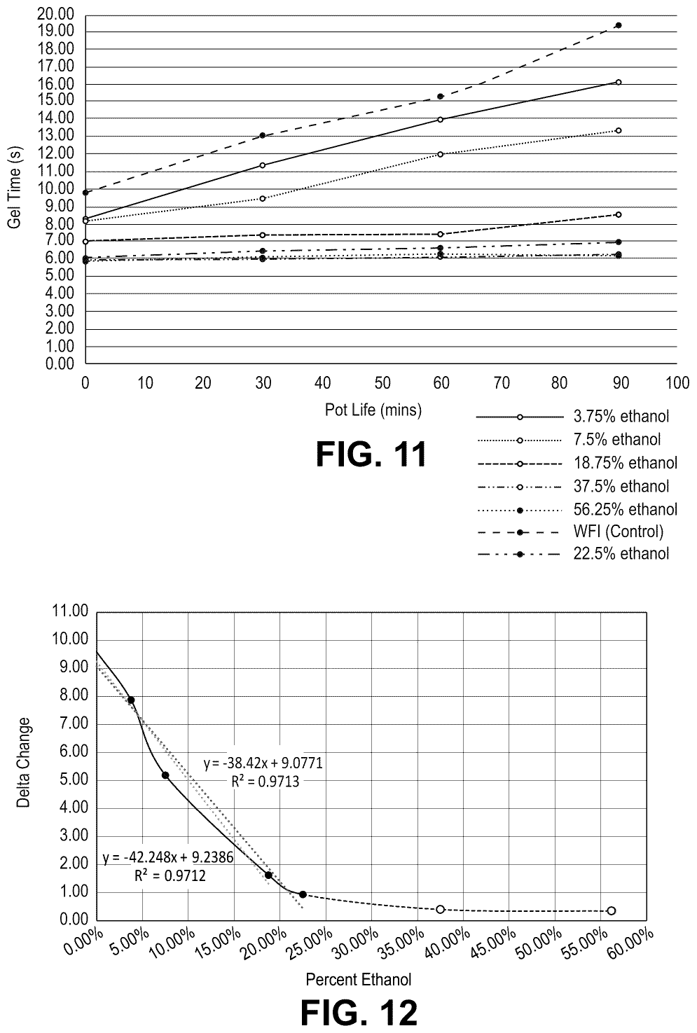

A set of precursor formulations were prepared from a first precursor and a second precursor, both as dry powders, a solvent composition, and a trace amount of FD&C Blue #1 to form a series of precursor formulations having a precursor concentration of 10% w/w, according to Table 1. The solvent composition was either unbuffered water-for-injection (WFI, pH 5.5) or an alcohol/WFI solution. The precursor formulation prepared with WFI solvent alone was used as a control. The precursor formulations were blue in color. For each precursor formulation, the first precursor was an eight-armed polyethylene glycol-based precursor having a 15,000 Da molecular weight and succinimidyl glutarate (SG) functional end groups (8A15k PEG SG, Jenkem USA). The second precursor was an eight-armed polyethylene glycol-based precursor having a 20,000 Da molecular weight and HCl-salted amine functional end groups (8A20k PEG amine-HCl, Jenkem USA). The first and second precursor were used in a 3:4 weight ratio in order to provide an approximately equivalent number of SG and amine functional end groups in the precursor formulations. The precursors dissolved rapidly in the solvent composition used to prepare each precursor formulation, as determined by the absence of particulates upon visual inspection. Immediately after dissolution, an aliquot of each precursor formulation was mixed with an aliquot of an activator solution (pH 8.02) in a 3:1 volume ratio (precursor to activator) to provide an activated formulation having a volume percent of alcohol as shown in Table 1. The activator solution was prepared by mixing 0.0438 M aqueous sodium borate decahydrate (Borax) and 0.2 M aqueous sodium phosphate dibasic. The alcohol concentration of the activated formulations ranged from zero to 56.25 vol %. The time to visualization of precipitates was measured and recorded as the gel time at a delay time of zero. The delay time (or “pot life”) is the time between preparation of the precursor formulation and mixing with the activator solution. The gel time is the time period between activation of the precursor formulation and formation of a blue-colored, solid to semi-solid mass due to crosslinking of the precursors leading to gel formation. There was no observable residual fluid post gelation, thus the volume percent of alcohol in the resulting hydrogel was determined to have the same volume percent alcohol as the activated formulation. At 30 minutes, 60 minutes, and 90 minutes after dissolution to form the precursor solutions, an aliquot of each precursor formulation was mixed with an aliquot of the activator solution in a 3:1 volume ratio to form a series of activated formulations from precursor formulations with various pot lives. The average of two gel time measurements for each activated formulation and pot life is shown in Table 2. FIG. 11 presents the data in Table 2 by plotting gel time as a function of pot life for each activated formulation. FIG. 12 is a plot of the overall change in gel time as a function of percent ethanol for each activated formulation shown in Table 2.

The results show that the gel time for each of the alcohol-containing precursor formulations was faster than the gel time for the WFI control sample at each pot life. The gel time was also shown to decrease with increasing alcohol concentration. The highest volume % alcohol formulation had a gel time of less than 6 seconds at a pot life of zero, which was about 41% faster than the gel time for the alcohol-free (WFI) formulation at a pot life of zero. An increase in gel time with pot life was seen for all of the formulations, however, the alcohol-containing precursor formulations were shown to be generally more tolerant to increasing pot life than the alcohol-free (WFI) formulations. After a delay time of 90 minutes, the highest vol % alcohol formulation had a gel time that was 68% faster than the comparative WFI formulation, which is a larger difference than the 41% faster result, noted above, at a pot life of zero. The stability of gel time as a function of pot life generally increased with the volume percent alcohol, with the two highest alcohol concentrations each showing gel time increases of only about 6-7% after 90 minutes of pot life. In comparison, the WFI formulation showed a gel time increase of about 99% over the same delay period.

This study showed that alcohol-loaded hydrogels, having up to about 56 volume % alcohol, can be prepared from two-part liquid formulations that gel in less than about 6-9 seconds, with the formulations being relatively stable after 90 minutes, such that all the alcohol-based formulations tested gelled in about 16 seconds or less.

TABLE 1

| | | Activated Formulation/ |
|---|---|---|
| | | Hydrogel Composition, |
| Sample | Solvent Composition | Volume % Ethanol |
| 1.1 | WFI | 0% |
| 1.2 | 5/95 v/v Ethanol/WFI | 3.75% |
| 1.3 | 10/90 v/v Ethanol/WFI | 7.5% |
| 1.4 | 25/75 v/v Ethanol/WFI | 18.75% |
| 1.5 | 30/70 v/v Ethanol/WFI | 22.5% |
| 1.6 | 50/50 v/v Ethanol/WFI | 37.5% |
| 1.7 | 75/25 v/v Ethanol/WFI | 56.25% |

TABLE 2

| | Gel Time, seconds | | | | | | |
|---|---|---|---|---|---|---|---|
| Pot Life, | | 3.75% | 7.5% | 18.75% | 22.5% | 37.5% | 56.25% |
| minutes | WFI | Ethanol | Ethanol | Ethanol | Ethanol | Ethanol | Ethanol |
| 0 | 9.72 | 8.19 | 8.16 | 6.88 | 5.97 | 5.79 | 5.78 |
| 30 | 13.00 | 11.38 | 9.44 | 7.35 | 6.41 | 5.97 | 6.00 |
| 60 | 15.28 | 13.97 | 12.00 | 7.41 | 6.60 | 6.06 | 6.19 |
| 90 | 19.31 | 16.06 | 13.35 | 8.50 | 6.91 | 6.19 | 6.13 |

### Example 2: Formulation Study

This example demonstrates that the in vitro performance of alcohol-loaded hydrogel precursor formulations can be adjusted to achieve target ranges of gel time, alcohol concentration, and modulus.

A set of precursor formulations were prepared from a first precursor and a second precursor, both as dry powders, a solvent composition, and a trace amount of FD&C Blue #1 to form a series of precursor formulations having a precursor concentration of 10% w/w, according to Table 3. The first and second precursor were used in a weight ratio that provided an approximately equivalent number of SG and amine functional end groups in the precursor formulations. For all but Samples 2a and 2c, the solvent composition was a 90/10 v/v ethanol/WFI solution. Sample 2a was prepared with 100% ethanol, but the precursors did not dissolve fully in the pure ethanol. Since the precursors did not fully dissolve, no activated formulation was formed with Sample 2a. The precursor formulations were blue in color. For all but Sample 2c, the first precursor and the second precursor were the same as described in Example 1. For Sample 2c, the first precursor was the same as described in Example 1, but the second precursor was trilysine acetate. For Sample 2c, the solvent was 80 vol % ethanol to get the trilysine to dissolve. Sample 2c formed a very loose and sticky hydrogel with a relatively long gel time. In contrast, Sample 2b quickly formed a very firm hydrogel. Based on the preliminary findings from Samples 2a-2c, the remaining study was performed with the solvent composition (90/10 v/v ethanol/water) and precursors used in Sample 2b.

Three sets of samples were tested, each set having a different volume ratio of precursor formulation to activator solution, as shown in Table 3. Within each set, the pH of the activator solution was varied. Each activator solution was prepared by mixing 0.0438 M aqueous sodium borate decahydrate (Borax) and 0.2 M aqueous sodium phosphate dibasic in a volume percent to provide the target pH. The precursors dissolved rapidly in the solvent composition used to prepare each precursor formulation, as determined by the absence of particulates upon visual inspection. Immediately after dissolution, an aliquot of each precursor formulation was mixed with an aliquot of the activator solution to provide an activated formulation having a volume percent of alcohol as shown in Table 3. The time to visualization of precipitates was measured in triplicate and recorded in Table 4 along with the average gel time for each sample. The pot life of Sample 2.1f was tested in 30-minute increments over a period of 2 hours and the results are shown in Table 5. The gel time increased by only about 5% over the 2-hour testing period. The results indicate that the precursor formulation is stable over a period of at least 2 hours.

TABLE 3

| | | | | | Volume Ratio | Activated |
|---|---|---|---|---|---|---|
| | | | | | of Precursor | Formulation/ |
| | | | | Activator | Formulation | Hydrogel |
| | First | Second | Solvent, | Solution | to Activator | Composition, |
| Sample | Precursor | Precursor | Ethanol/WFI | pH | Solution | Volume % Ethanol |
| 2a | 8A15kPEG | 8A20kPEG | 100% Ethanol | N/A | N/A | N/A* |
| | SG | amine-HCl | | | | |
| 2b | 8A15kPEG | 8A20kPEG | 90/10 v/v | 8.02 | 4:1 | 72%** |
| | SG | amine-HCl | | | | |
| 2c | 8A15kPEG | Trilysine | 80/20 v/v | 8.02 | 4:1 | 64%*** |
| | SG | Acetate-HCl | | | | |
| 2.1a | 8A15kPEG | 8A20kPEG | 90/10 v/v | 8.02 | 4.1 | 72% |
| | SG | amine-HCl | | | | |
| 2.1b | 8A15kPEG | 8A20kPEG | 90/10 v/v | 9.97 | 4:1 | 72% |
| | SG | amine-HCl | | | | |
| 2.1c | 8A15kPEG | 8A20kPEG | 90/10 v/v | 9.79 | 4:1 | 72% |
| | SG | amine-HCl | | | | |
| 2.1d | 8A15kPEG | 8A20kPEG | 90/10 v/v | 9.53 | 4:1 | 72% |
| | SG | amine-HCl | | | | |
| 2.1e | 8A15kPEG | 8A20kPEG | 90/10 v/v | 8.81 | 4:1 | 72% |
| | SG | amine-HCl | | | | |
| 2.1f | 8A15kPEG | 8A20kPEG | 90/10 v/v | 9.06 | 4:1 | 72% |
| | SG | amine-HCl | | | | |
| 2.2a | 8A15kPEG | 8A20kPEG | 90/10 v/v | 8.02 | 2:1 | 60% |
| | SG | amine-HCl | | | | |
| 2.2b | 8A15kPEG | 8A20kPEG | 90/10 v/v | 9.97 | 2:1 | 60% |
| | SG | amine-HCl | | | | |
| 2.3a | 8A15kPEG | 8A20kPEG | 90/10 v/v | 8.02 | 1:1 | 45% |
| | SG | amine-HCl | | | | |
| 2.3b | 8A15kPEG | 8A20kPEG | 90/10 v/v | 9.97 | 1:1 | 45% |
| | SG | amine-HCl | | | | |

*The precursors did not dissolve in 100% ethanol.
**A very firm hydrogel was quickly formed.
***A very loose and sticky gel was slowly formed.

TABLE 4

| | | | | Avg. Gel |
|---|---|---|---|---|
| Sample | Measured Gel Time, seconds | | | Time, sec. |
| 2.1a | 13.37 | 12.63 | 14.56 | 13.5 |
| 2.1b | 1.81 | 1.63 | 1.62 | 1.7 |
| 2.1c | 1.69 | 1.81 | 1.82 | 1.8 |
| 2.1d | 1.94 | 2.06 | 2.06 | 2.0 |
| 2.1e | 4.94 | 5.50 | 5.31 | 5.3 |
| 2.1f | 3.32 | 2.93 | 2.94 | 3.1 |
| 2.2a | 4.13 | 4.75 | 4.56 | 4.5 |
| 2.2b | 1.06 | 1.06 | 1.00 | 1.0 |
| 2.3a | 4.63 | 4.31 | 4.63 | 4.5 |
| 2.3b | 0.93 | 0.94 | 1.00 | 1.0 |

TABLE 5

| Pot Life, minutes | Average Gel Time, sec. |
|---|---|
| 0 | 3.7 |
| 30 | 3.7 |
| 60 | 3.7 |
| 90 | 3.9 |
| 120 | 3.8 |

The results showed that precursor composition, activator solution pH, and volume ratio of precursor formulation to activator solution can be adjusted to tailor the gel time, the alcohol-loading, and the modulus of the hydrogel. The tested formulations had gel times ranging from 1.0 seconds to 13.5 seconds. Hydrogels with an alcohol-concentration up to 72% were prepared, with gel times ranging from 1.7 seconds to 13.5 seconds. A formulation for preparing a 72% alcohol-loaded hydrogel via a gel time of about 3.1 seconds was identified. The pot life results suggest that the precursor formulations would be stable enough to provide predictable gel time performance for at least 8 hours after preparation.

### Example 3: Modulus and Persistence Study

This example demonstrates the effect of precursor composition on the modulus and persistence of an alcohol-loaded hydrogel.

A set of precursor formulations were prepared from a first precursor and a second precursor, both as dry powders, a solvent composition, and a trace amount of FD&C Blue #1 to form a series of precursor formulations having a precursor concentration of 10% w/w, according to Table 6. The three precursor formulations were the same with the exception of the first precursor composition. Formulation 3.1 used an 8 arm 15K PEG with succinimidyl glutarate (SG) functional end groups. Formulation 3.2 used an 8 arm 15K PEG with succinimidyl succinate (SS) functional end groups. Formulation 3.3 used a 4 arm 20K PEG with succinimidyl glutarate (SG) functional end groups.

TABLE 6

| | | | | | Volume Ratio | Activated |
|---|---|---|---|---|---|---|
| | | | | | of Precursor | Formulation/ |
| | | | | Activator | Formulation | Hydrogel |
| | First | Second | Solvent, | Solution | to Activator | Composition, |
| Formulation | Precursor | Precursor | Ethanol/WFI | pH | Solution | Volume % Ethanol |
| 3.1 | 8A15kPEG | 8A20kPEG | 90/10 v/v | 9.06 | 4:1 | 72% |
| | SG | amine-HCl | | | | |
| 3.2 | 8A15kPEG | 8A20kPEG | 90/10 v/v | 8.50 | 4:1 | 72% |
| | SS | amine-HCl | | | | |
| 3.3 | 4A20kPEG | 8A20kPEG | 90/10 v/v | 9.96 | 4:1 | 72% |
| | SG | amine-HCl | | | | |

For Formulations 3.1 to 3.3, a 3.0 ml aliquot of the precursor formulation was drawn into a first syringe. Aqueous activator solutions were prepared by mixing 0.0438 M sodium borate decahydrate (Borax) and 0.2 M sodium phosphate dibasic. The pH of the activator solution used for each formulation was chosen to achieve a target gel time of approximately 3 seconds. The activator solution pH for formulations 3.1 to 3.3 was 9.06, 8.50, and 9.96, respectively. For each formulation, a 0.75 ml aliquot of the activator solution was drawn into a second syringe. A Y connector with two luer lock connections was attached to a ¼″ tube adaptor to luer lock fitting, and the tube adaptor attached to ¼″ ID clear silicone tubing (Silastic®) containing a static mixing element proximal to the Y connector. The first syringe had a first diameter R and the second syringe had a smaller diameter r, such that the ratio of r/R was equal to 0.5, the square root of the inverse of the volume ratio. The first syringe and the second syringe were attached to the Y connector and a plunger cap was added to the ends of the syringes to ensure equal volume deployment from the two syringes. FIG. 1A depicts one embodiment of an asymmetric syringe assembly as described above. The syringes were deployed, injecting material into the length of the silicone tubing. Initial gelation was observed to occur between 3-5 seconds, as evidenced by air bubble movement within the tubing. Samples were allowed to continue to gel for a period of time that was more than 20 times the initial gelation time (greater than 2.5 minutes) to ensure full cure.

Using a razor blade, the silicone tubing containing each gel was cut into ten uniform lengths and gel samples (approximately 0.2 g) were removed from each length. Gel samples were placed in a phosphate-buffered saline solution (PBS) having a selected volume corresponding with a target dilution ratio of 250:1. The term dilution ratio is the ratio of the mass of the PBS to the gel sample mass. A dilution ratio was used as a way to model diffusion of fluids to and from an in vivo uterine location. The pH of PBS at 50° C. was measured as 7.57.

The modulus of each gel sample was measured as a function of time at 50° C. (accelerated conditions) in PBS and/or at 37 C ° ° C. (real-time conditions) in PBS. Once the gel samples began to lose shape and modulus was not able to be tested, the gel samples were observed for persistence. Modulus was able to be measured in the range of 5 kPa to 200 kPa. The gel samples were also checked regularly until they were no longer visually detectable.

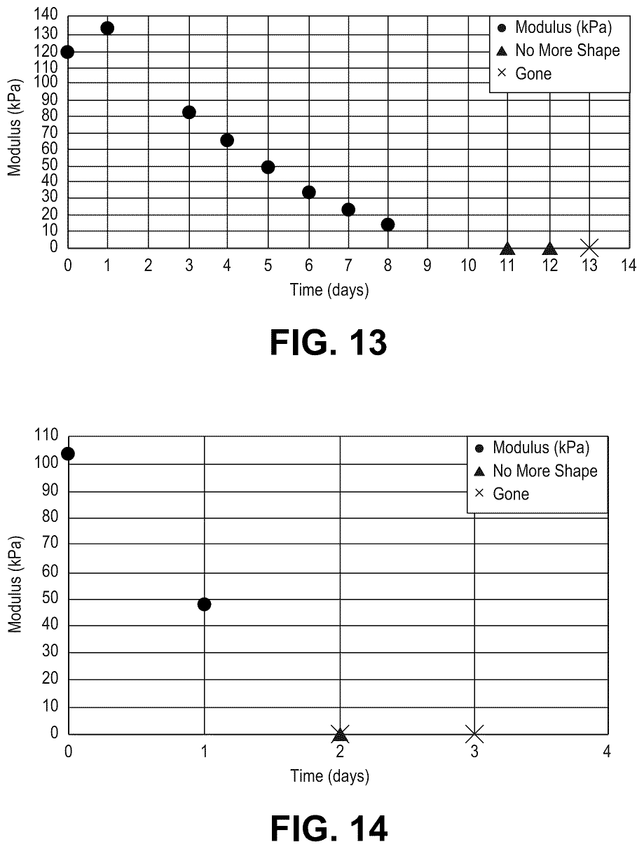

The gel samples from Formulation 3.1 were tested under accelerated conditions. The samples lost shape between day 8 and day 11. All of the gel samples from Formulation 3.1 disappeared by day 13. FIG. 13 is a plot of the modulus of the gel samples from Formulation 3.1 as a function of time in (accelerated) days. A loss of shape may roughly correspond with timing expected for expulsion from a patient's uterus, but significant patient variability would be expected in practice due to variation in health and physiology of a particular patient. The results show that the modulus initially increased from a value of about 120 kPa at the time of formation of the gel to about 133 kPa after about 1 day. Thereafter, the modulus decreased to about 13 kPa after 8 days. At days 11 and 12 the modulus was not able to be tested due to the gel samples having lost their shape. At day 13, the gel samples were no longer visible. The results suggest that the loss of shape of the gel samples from Formulation 3.1 would be at about day 24 to day 33 in real-time, with disappearance by (real-time) day 39. For intrauterine use, the hydrogel is expected to be expelled by natural uterine contractions once the hydrogel loses shape. So for intrauterine applications, loss of shape can be considered the end of treatment time since expulsion is expected. For other placements, loss of shape may or may not correspond with elimination of the hydrogel from the patient. Generally, though the ablation composition is essentially depleted from the hydrogel prior to degradation of the hydrogel for removal from the body so that there is no sudden release of a bolus of ablation composition.

The gel samples from Formulation 3.2 were initially tested under accelerated conditions, however, after 1 day the gel samples had all disappeared. As a result, the testing of the gel samples from Formulation 3.2 were instead tested under real-time conditions. FIG. 14 is a plot of the modulus of the gel samples from Formulation 3.2 as a function of time in (real-time) days. The results show that the modulus at the time of formation of the gel was about 103 kPa. The modulus decreased to about 48 kPa after 1 day. Some of the gel samples had disappeared after 2 days, with the remaining samples having lost shape. All of the gel samples had disappeared before day 3.

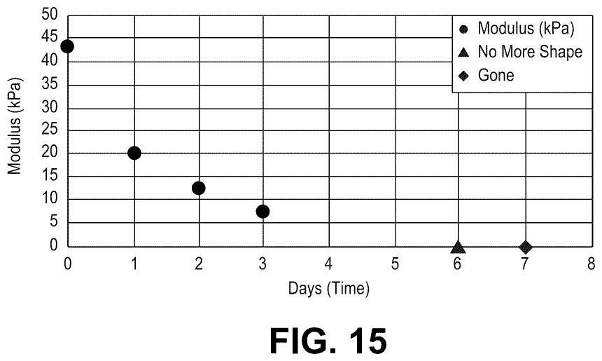

The gel samples from Formulation 3.3 were tested under accelerated conditions. The samples lost shape between day 3 and day 6. All of the gel samples from Formulation 3.3 disappeared by day 7. FIG. 15 is a plot of the modulus of the gel samples from Formulation 3.3 as a function of time in (accelerated) days. The results show that the modulus at the time of formation of the gel was about 43 kPa. The modulus decreased to about 20 kPa after 1 day. Thereafter, the modulus decreased to about 8 kPa after 3 days. At day 6, the modulus was not able to be tested due to the gel samples having lost their shape. At day 7, the gel samples were no longer visible. The results suggest that the loss of shape of the gel samples from Formulation 3.3 would be at about day 9 to day 18 in real-time, with disappearance by (real-time) day 21.

These results show that the modulus and persistence profiles depend on the ester precursor composition. Extrapolation of the results of this study suggest that hydrogel formulations can prepared to provide a targeted range of the persistence of an alcohol-loaded hydrogel over a period of about 3 days to about 39 days. The results suggest that blended formulations may also be useful for further tailoring of the alcohol-loaded hydrogel modulus and persistence.

### Example 4: Ex-Vivo Model Uterus Benchtop Study

This example illustrates the use of an in-situ formed alcohol-loaded hydrogel to perform chemoablation of a pig cervix. The pig cervix was used as a model of a human uterus. The hydrogel was 82.25% alcohol by volume.

In this example, a previously frozen, ex-vivo pig cervix was used. The distal end of the cervix was connected to an approximately 2-inch section of the uterine horn bifurcation. The cervix was brought to room temperature over a period of about 24 hours prior to the study.

A delivery system was assembled according to the illustration in FIG. 1A. Syringe 115 had a marked volume capacity of 12 ml, and syringe 117 had a marked volume capacity of 1 ml, with the diameters of syringe 115 and syringe 117 in a ratio to provide a 11:1 volume ratio of the precursor formulation to the activator solution into catheter 104 upon deployment of plunger cap 120. An ester-amine precursor formulation was prepared by mixing 8a15K PEG SG and 8A20kPEG amine-HCl in a 3:4 weight ratio, to provide an approximately equivalent number of reactive amines to reactive ester end groups and dissolving the mixture in a 90/10 v/v ethanol/WFI solvent composition. The WFI had a pH of about 5.5. An 8.8 ml aliquot of the ester-amine precursor formulation was drawn into the first syringe. An activator solution (pH 10.92) was prepared by mixing 0.0438 M sodium borate decahydrate (Borax) and 0.2 M sodium phosphate dibasic. A 0.83 ml aliquot of the of activator solution was drawn into the second syringe. The precursor formulation was colored with a dilute concentration of FD&C blue #1.The activator solution was uncolored.

Catheter 104 was connected to the Y-connector and syringe assembly via fitting 111. Catheter 104 was approximately 10 inches in length. The syringe assembly included syringe holder 118 and syringes 115 and 117. Catheter 104 was inserted into the cervix using the external length as a guide to provide an approximately 1 cm spacing between distal end of installation tip 102 and the distal end of the cervix during the installation of the hydrogel. The plunger of syringe holder 118 was pressed to fully deploy the precursor formulation and the activator solution from their respective syringes. Next, the catheter was pulled out from the cervix. All of the installed fluid remained in the cervix after delivery. There was no evidence of the installed hydrogel being expelled from the cervix.

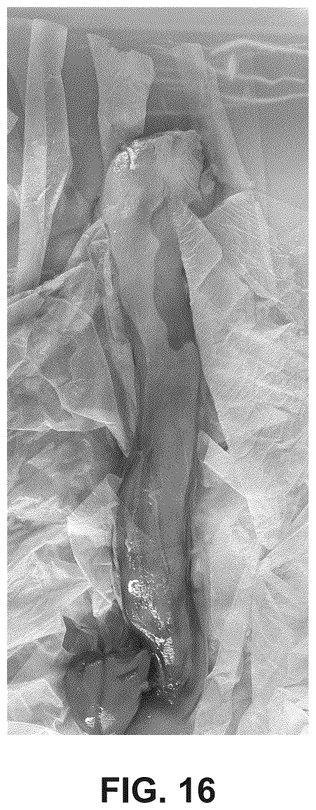

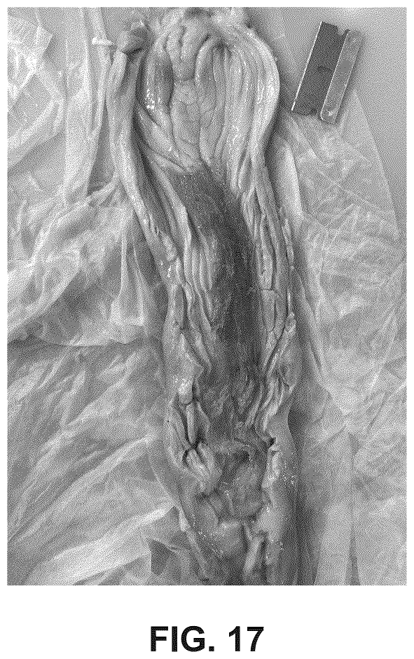

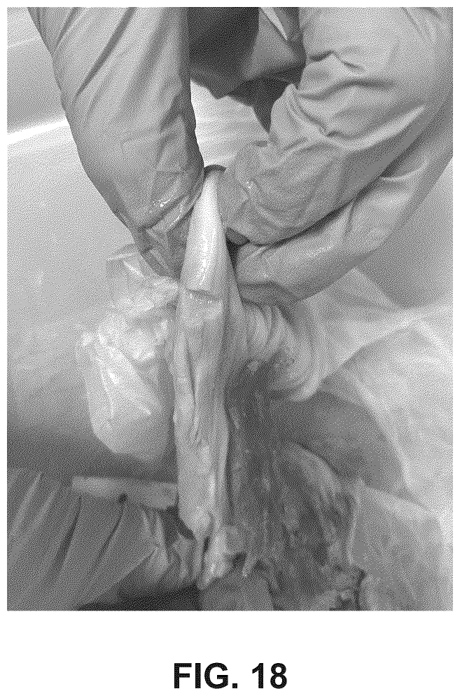

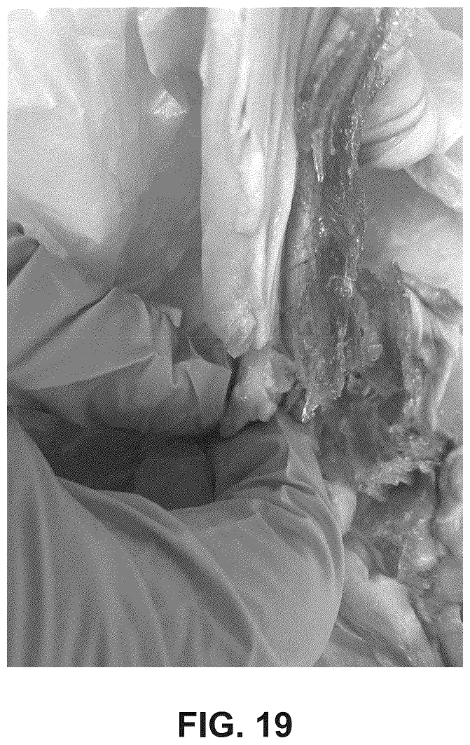

The filled cervix was covered with Kimwipes and saline for about four hours. FIG. 16 is a photograph of the cervix after the four hours. After the four hours, the cervix was sectioned along the sagittal plane. A continuous hydrogel was observed that completely filled the cervical cavity. The solid hydrogel was removed, and it was noted that it held its shape after removal. As shown in FIG. 17, the cervix showed blue staining along the entire length of the cervical cavity. Blue staining was also observed to have penetrated into the cervical walls. The blue staining reveals the tissue that was contacted by the ethanol-loaded hydrogel or the ethanol that diffused out of the hydrogel. FIGS. 18 and 19 are photographs showing healthy (non-ablated) cervix side wall tissue and ablated cervix side wall tissue, respectively.

The results of this study showed that the alcohol-loaded hydrogel was effective at delivering alcohol to a cervical cavity and selectively ablating cervical tissue. The hydrogel filled the distal volume of the cervical cavity and was firm enough to separate and tamponade the cervical walls and not be ejected at the end of the installation procedure. The results suggest that alcohol-loaded hydrogel could be effective for use in performing gentle chemoablation in an in-vivo human uterus.

### Example 5: In-Vivo Animal Study

This example demonstrates the efficacy of an in-situ formed alcohol-loaded hydrogel to perform in vivo chemoablation of a rabbit uterus. Performance of the alcohol-loaded hydrogel is compared to the performance of an alcohol solution. The hydrogel comprised liquid that was 73% alcohol by volume.

### Part A. Test and Control Procedures

In this study, three female New Zealand White rabbits were used. The Group 1 animals (Animal 1, 8 months old) and Animal 2, 7 months old) each underwent chemical ablation to the right and the left uterine horn using an alcohol-loaded hydrogel composition on Day 0. The Group 2 animal (Animal 3, 7 months old) was used for control testing, with animal 3 undergoing chemical ablation to the right uterine horn using an alcohol solution on Day 0. The left uterine horn of animal 3 was untreated. Both the fluid in the alcohol-loaded hydrogel composition and the alcohol solution were 73 vol % ethanol. Table 7 summarizes the study protocol.

TABLE 7

| | Animal | Description of | Treatment | | Necropsy |
|---|---|---|---|---|---|
| Group | Number | Procedure | Composition | Location | Time Point |
| 1 | 1, 2 | Chemical Ablation | Alcohol-loaded | Right and | Day 7 |
| | | of Bilateral Uterine | Hydrogel | Left Horns | |
| | | Horn (Day 0) | Composition | | |
| | | | (73 vol % ethanol | | |
| | | | hydrogel) | | |
| 2 | 3 | Chemical Ablation | Aqueous Alcohol | Right Horn | |
| (control) | | of Unilateral Uterine | Solution | | |
| | | Horn (Day 0) | (73 vol % ethanol | | |
| | | | solution) | | |
| | 3 | Untreated | N/A | Left Horn | |

For the Group 1 animals, the chemical ablation of each horn was performed using 1.0 mL of the alcohol-loaded hydrogel composition. The alcohol-loaded hydrogel composition was delivered to the right horn using a delivery system as described in Example 4, except the capacity of syringe 115 and syringe 117 were adjusted to the smaller volume of the rabbit horn, catheter 104 was a needle, and the diameters of the syringes were in a ratio to provide a 11:1 volume ratio of the precursor formulation to the activator solution into the needle upon deployment of plunger cap 120. Thereafter, a 0.8 ml aliquot of the ester-amine precursor formulation was drawn into a first syringe. A 0.2 ml aliquot of the of activator solution was drawn into a second syringe. As in the delivery system of Example 4, the precursor formulation was colored with a dilute concentration of FD&C blue #1. The activator solution was uncolored.

A midline incision was made to the rabbit and the uterine horns exposed. The needle was connected to the Y-connector and syringe assembly via fitting 111. The needle was a 21 gauge needle with a length of approximately 1.5 inches. The syringe assembly included syringe holder 118 and syringes 115 and 117. The needle was inserted into the right uterine horn via a small incision in the rabbit uterine horn to provide an approximately 1 cm spacing between distal end of installation tip 102 and the distal end of the uterine horn during the installation of the hydrogel. The plunger of syringe holder 118 was pressed to fully deploy the precursor formulation and the activator solution from their respective syringes. Then, the needle was pulled out from the right uterine horn. The above hydrogel delivery procedure was repeated for the left horn, which was accessed through the left uterine horn. The above modified hydrogel delivery procedure was then repeated for the second animal from Group 1. All of the installed fluid remained in the horn after delivery. Additionally, it was observed that each of the horns in Group 1 were visibly distended (swollen) after installation of the hydrogel.

For the Group 2 animal, the chemical ablation of a single horn was performed using 1.0 mL of the alcohol solution. The alcohol solution was prepared by mixing ethanol and WFI in a 73/27 v/v ratio. The WFI had a pH of about 5.5. The alcohol solution was colored with a dilute concentration of FD&C blue #1. The alcohol solution was drawn into a syringe and the syringe was connected to a needle via a luer fitting. The needle was the same type and length as used in the alcohol-loaded hydrogel delivery for Group 1.

As with the Group 1 animals, a midline incision was made to Animal 3 and the uterine horns exposed. The needle was inserted into the right uterine horn to provide an approximately 1 cm spacing between distal end of the installation tip and the distal end of the uterine horn during the installation of the alcohol solution. The plunger of the syringe was pressed to fully deploy the alcohol solution from the syringe. Then, the needle was pulled out from the right horn. All of the installed alcohol solution remained in the right horn after delivery. In particular, there was no observation of liquid refluxing through the injection site or out of the vaginal canal. It was also observed that, unlike the Group 1 hydrogel installation, there was no visible distention of the horn after installation of the alcohol solution. The left horn was left untreated.

### Part B. Evaluation Procedures and Results

Clinical pathology was assessed and body weights with a body condition score were recorded prior to the assigned procedure and prior to necropsy. Animals lost weight (−4%, −4%, and −3%, for Animals 1-3, respectively) but maintained body condition over the course of the study. Clinical observations were performed daily. Animal 1 was observed to have some red-colored urine as well as suspected test material present in the cage's pan liner on Days 1 and 2. Animal 3 also had red-colored urine present on Day 4. The animals were euthanized on Day 7, a limited necropsy performed, and the treatment sites were fixed/collected in 10% formalin, paraffin processed, and stained with hematoxylin and eosin (H&E) for histomorphologic evaluation.

Inflammation was only observed in Group 1 and when present was characterized by the presence of heterophils and macrophages in both animals in this group. Table 8 summarizes the mean±standard deviation, the median, and the percent incidence of inflammation and inflammatory cell types as observed on Day 7 during necropsy.

TABLE 8

| | Day 7 | | | | | |
|---|---|---|---|---|---|---|
| | Group 1 | | Group 2 | | | |
| | 73 vol % ethanol | | 73 vol % ethanol | | Group 2 | |
| Parameter | hydrogel (n = 4) | | solution (n = 1) | | Untreated (n = 1) | |
| Inflammation | 1.75 ± 0.32 | 100% | 0.00 ± NA | 0% | 0.00 ± NA | 0% |
| | 1.84 | | 0.00 | | 0.00 | |
| Heterophils | 1.33 ± 0.47 | 100% | 0.00 ± NA | 0% | 0.00 ± NA | 0% |
| | 1.17 | | 0.00 | | 0.00 | |
| Eosinophils | 0.00 ± 0.00 | 0% | 0.00 ± NA | 0% | 0.00 ± NA | 0% |
| | 0.00 | | 0.00 | | 0.00 | |
| Macrophages | 1.17 ± 0.64 | 100% | 0.00 ± NA | 0% | 0.00 ± NA | 0% |
| | 1.34 | | 0.00 | | 0.00 | |
| Lymphocytes | 0.00 ± 0.00 | 0% | 0.00 ± NA | 0% | 0.00 ± NA | 0% |
| | 0.00 | | 0.00 | | 0.00 | |
| Giant Cells | 0.00 ± 0.00 | 0% | 0.00 ± NA | 0% | 0.00 ± NA | 0% |
| | 0.00 | | 0.00 | | 0.00 | |
| Foamy Macrophages | 0.00 ± 0.00 | 0% | 0.00 ± NA | 0% | 0.00 ± NA | 0% |
| | 0.00 | | 0.00 | | 0.00 | |

NA = Not Applicable.
General Observations Scoring Matrix:
0 = No response; 1 = Minimal/focal/barely detectable; 2 = Mild/focal or rare multifocal/slightly detectable; 3 = Moderate/multifocal to confluent/easily detectable; 4 = marked/diffuse/overwhelming presence.

Table 9 summarizes the mean±standard deviation (top number), the median (lower number), and the percent incidence of general observations on Day 7 during necropsy (percentage values) based on pathologist scoring using the indicated semi-quantitative scoring matrix. Sections of both right and left uterine horns in Animals 1 and 2 (Group 1) had decreased to absent endometrial glands while normal endometrial glands were detected in Animal 3.

TABLE 9

| | Day 7 | | | | | |
|---|---|---|---|---|---|---|
| | Group 1 | | Group 2 | | | |
| | 73 vol % ethanol | | 73 vol % ethanol | | Group 2 | |
| Parameter | hydrogel (n = 4) | | solution (n = 1) | | Untreated (n = 1) | |
| Fibrosis | 0.00 ± 0.00 | 0% | 0.00 ± NA | 0% | 0.00 ± NA | 0% |
| | 0.00 | | 0.00 | | 0.00 | |
| Mineralization | 0.00 ± 0.00 | 0% | 0.00 ± NA | 0% | 0.00 ± NA | 0% |
| | 0.00 | | 0.00 | | 0.00 | |
| Fibroplasia/Reactive | 1.00 ± 0.47 | 100% | 0.00 ± NA | 0% | 0.00 ± NA | 0% |
| Fibroblasts | 0.84 | | 0.00 | | 0.00 | |
| Hemorrhage | 0.58 ± 0.32 | 100% | 0.00 ± NA | 0% | 0.00 ± NA | 0% |
| | 0.50 | | 0.00 | | 0.00 | |
| Necrosis | 0.00 ± 0.00 | 0% | 0.00 ± NA | 0% | 0.00 ± NA | 0% |
| | 0.00 | | 0.00 | | 0.00 | |
| Neovascularization | 0.08 ± 0.17 | 25% | 0.00 ± NA | 0% | 0.00 ± NA | 0% |
| | 0.00 | | 0.00 | | 0.00 | |
| Endometrial | 1.00 ± 0.86 | 75% | 0.00 ± NA | 0% | 0.00 ± NA | 0% |
| Ulceration Erosion | 1.00 | | 0.00 | | 0.00 | |
| Endometrial Edema | 0.84 ± 0.58 | 100% | 0.00 ± NA | 0% | 0.00 ± NA | 0% |
| | 0.67 | | 0.00 | | 0.00 | |
| Uterine Luminal | 0.25 ± 0.50 | 25% | 0.00 ± NA | 0% | 0.00 ± NA | 0% |
| Dilation | 0.00 | | 0.00 | | 0.00 | |
| Residual Test/Control | 0.75 ± 0.57 | 75% | 0.00 ± NA | 0% | 0.00 ± NA | 0% |
| Article | 0.84 | | 0.00 | | 0.00 | |

NA = Not Applicable.
General Observations Scoring Matrix:
0 = No response; 1 = Minimal/focal/barely detectable; 2 = Mild/focal or rare multifocal/slightly detectable; 3 = Moderate/multifocal to confluent/easily detectable; 4 = marked/diffuse/overwhelming presence.

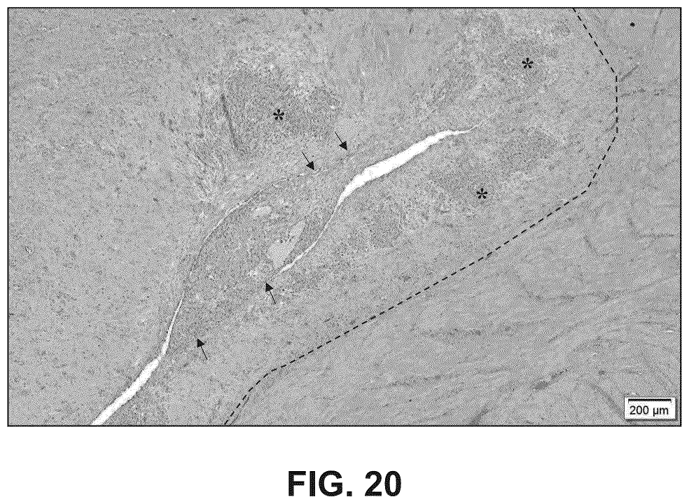

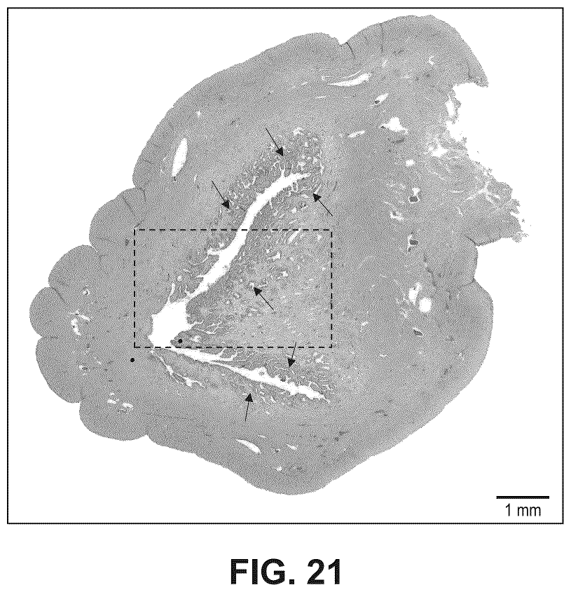

FIG. 20 shows a stained section of the right uterine horn of Animal 2 (treated with 73vol % ethanol hydrogel). The arrows in FIG. 20 indicate suspected endometrial adhesions within two sections (cranial and mid sections) associated with areas of endometrial ulceration/erosions. FIG. 20 also shows that the myometrium (below the dashed line) and the endometrium (above the dashed line) were absent visible endometrial glands. Asterisks in FIG. 20 indicate inflammatory infiltrate. FIG. 21 shows a stained section of the right uterine horn of Animal 3 (treated with 73 vol % ethanol solution). FIG. 21 is absent evidence of visible histological changes. Endometrial glands remain visible, as shown by the arrows.

This example shows that the alcohol-loaded hydrogel was effective at performing in vivo chemical ablation in a rabbit model without complications. Evidence of chemical endometrial ablation the alcohol-loaded hydrogel included a loss of endometrial glands (with certain regions having a total absence of glands), an inflammation response with the presence of heterophils and macrophages, an observation of fibroplasia/reactive fibroplasts, hemorrhage, neovascularization, endometrial ulceration/erosion, endometrial edema, and uterine dilation. Further evidence of the body's response to the alcohol-loaded hydrogel treatment was the partial expulsion of the installed hydrogel from one horn.

Significantly, the observation of suspected adhesions in the rabbit uterine horn strongly suggests ablative damage to the tissue, which is noteworthy given the known difficulty in forming adhesion in animal models. While adhesions in the relatively small diameter rabbit uterine horn do not suggest that adhesions would form in a human uterus during treatment with an alcohol-loaded hydrogel, the adhesions do suggest that the alcohol-loaded hydrogel would be effective at performing chemical ablation of human fallopian tubes for the purpose of sterilization based on the anatomical similarities between extended bifurcated uteri of the rabbit to the human fallopian tube.

The alcohol-loaded hydrogel significantly outperformed an alcohol solution having the same concentration of ethanol by volume. The alcohol solution treatment showed no impact relative to the untreated control. The results indicated minimal evidence of chemical ablation with the alcohol solution treatment. The results suggest that the extended dwell time provided by the installed hydrogel as compared to an alcohol solution enables a more effective chemical ablation while reducing the risk of alcohol ablation of off-target tissues. The results also suggest that the swollen fill achieved with the installed hydrogel provides pressure between the hydrogel and the endometrium which further promotes consistent contact of the surface of the endometrium with the alcohol as well as diffusion of the alcohol into the endometrium.

### Example 6: Ex-Vivo Uterus Benchtop Study

This example illustrates the use of an in-situ formed alcohol-loaded hydrogel to perform chemoblation of an excised human uterus. The hydrogel comprised a liquid phase with 73.4% alcohol by volume.

In this example, a set of post-hysterectomy, benign human uteri were obtained from pre-menopausal women according to standard medical research protocols. Each extirpated uterus was weighed. Then, within several minutes after removal, an alcohol loaded hydrogel was installed into the extirpated uterus.

For each extirpated uterus, the following protocol was followed. A delivery system was assembled according to the image in FIG. 1A, with catheter 104 being further provided with an egress limiter, according to the depiction of egress limiter 106 over catheter 192 shown in FIG. 4A. The diameters of syringe 115 and syringe 117 were in a ratio to provide a 4.5:1 volume ratio of the precursor formulation to the activator solution into catheter 104 upon deployment of plunger cap 120. An ester-amine precursor formulation was prepared by mixing 8a15kPEGSG and 8A20kPEG amine-HCl in a 3:4 weight ratio, to provide an approximately equivalent number of reactive amines to reactive ester end groups and dissolving the mixture in a 90/10 v/v ethanol/WFI solvent composition. The WFI had a pH of about 5.5. An 8.2 ml aliquot of the ester-amine precursor formulation was drawn into the first syringe. An activator solution (pH 10.92) was prepared by mixing 0.0438 M sodium borate decahydrate (Borax) and 0.2 M sodium phosphate dibasic. A 1.8 ml aliquot of the of activator solution was drawn into the second syringe. The precursor formulation was colored with a dilute concentration of FD&C blue #1. The activator solution was uncolored.

A uterine sound (Integra LifeSciences, product number 30-6000) was used to determine the fundal depth of the ex-vivo uterus. Then the uterine sound was placed along the assembly of catheter 104 and the egress limiter. The position of the cap element of the egress limiter was adjusted along the catheter using the uterine sound as a guide to provide an approximately 1 cm spacing between distal end of installation tip 102 and the fundus during the installation of the hydrogel. The catheter and egress limiter were connected to the Y-connector and syringe assembly via a luer fitting. The catheter was inserted into the uterus until the distal portion of the cap element entered the cervical canal and the proximal portion of the cap element was pressed against the external orifice of the cervix. A forceps was used to grasp the lip of the cervix to provide resistance during the insertion process. The delivery system was held by syringe holder 118 and firm pressure between the cervix and the cap element was applied while the plunger was pressed to fully deploy the precursor formulation and the activator solution from their respective syringes. Next, the catheter was pulled out from the cervix, leaving the egress limiter against the external orifice of the cervix. After approximately 2 seconds, the egress limiter was pulled away from the cervix. The uterus was again weighed. The increase in weight of each uteri after the installation of the installed hydrogel was about 6 grams. In each case, the majority of the hydrogel precursor remained in the uterus after delivery with generally a relatively small amount of the precursor outflowing. There was no evidence of the installed hydrogel being expelled from any of the uteri. Each uterus was placed in a plastic bag along with a saline-moistened gauze and the bag was sealed. The sample was allowed to acclimate at room temperature (23° C. to 26° C.) for about 4 hours. The acclimation period allowed for pre-existing remnant NADPH/NADH and non-effected enzymes to inactivate in cells destined to die at the outer effected tissue boundary. Without this acclimation period, such remnant NADPH/NADH and non-effected enzymes may have resulted in false positive staining and an underestimation of the maximum effected tissue depths.

After the four hours, each uterus was dissected in the midline coronal plane. The dissected halves were place in a TTC stain for about 30 minutes.

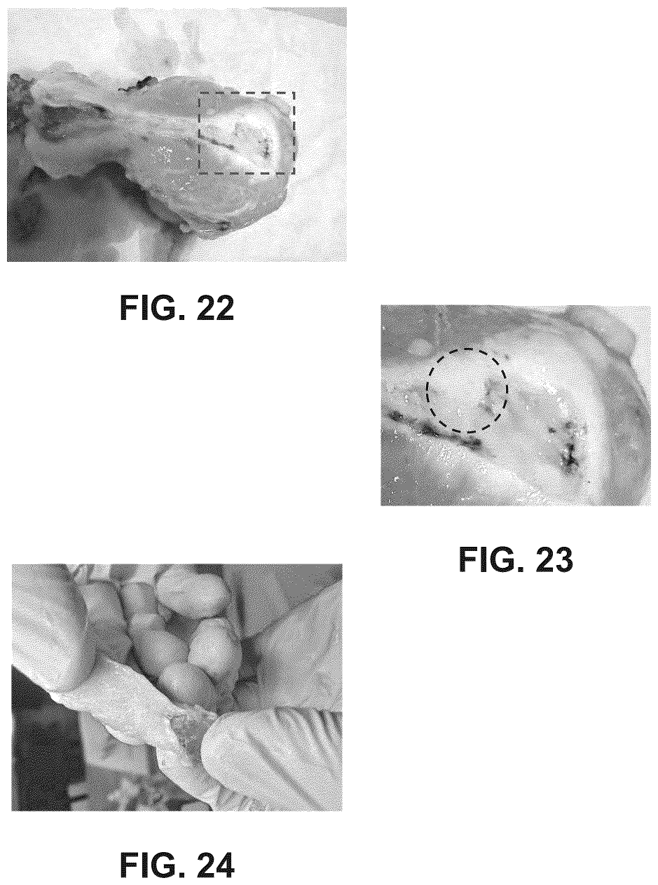

Ablation was evident on all test uteri and was consistent around the entire uterine surface. FIG. 22 also shows the consistency of the ablation as evidenced by the continuous cross-section of whitened tissue. The ablation depth was approximately 2-5 mm in all of the dissected uteri. FIG. 22 also shows that a continuous hydrogel that completely filled the uterine cavity including the cervical canal. This filling feature was observed for all test uteri. FIG. 22 also shows that the hydrogel had a gradient of intensity of blue coloration, with some regions of darker blue color and other regions of lighter blue color. The lighter-colored hydrogel is dye-voided. The increased thickness of the ablated margins adjacent to the lighter-colored hydrogel seem to suggest that the lighter-colored hydrogel is also alcohol-voided. These results also suggest a correlation between the diameter of the hydrogel in a given plane and the depth of ablation in that plane. In particular, from a semi-qualitative analysis, it appears that the zone of ablation was somewhat wider towards the fundus where most of the bulk material was deposited. As the uterine cavity narrows approaching the inner os, the zone of ablation appears to narrow. There seems to be a direct correlation between diameter of gel, total amount alcohol, and potential depth of effectiveness. By overinstalling/distending the uterine cavity, the total amount of installed hydrogel could be increased and it is expected that this would widen the narrow areas approaching the cervix to improve depth of ablation in those areas.

Furthermore, it was observed that the uteri depicted in FIG. 22 had an internal fibroid in the coronal wall. FIG. 23 shows that the alcohol-loaded hydrogel conformally filled around the fibroid. Ablation was relatively consistent around the entire uterine surface, including around the internal fibroid. Thus, it was observed that not only was the alcohol-loaded hydrogel ablation procedure robust enough to accommodate abnormalities, such as fibroids, alcohol-loaded hydrogel ablation could also be used to target fibroids and thereby potentially avoid the need for a hysterectomy. Injection of the alcohol-loaded hydrogel directly into a fibroid is contemplated.

This study further showed that the alcohol-loaded hydrogel adhered strongly to the uterine tissue, as demonstrated in FIG. 24. These results are in contrast to previous experimental work with installing comparable hydrogels without alcohol into the uterus. (See, for example, U.S. patent application 2022/0142653.) The present results suggest that the enhanced adhesion is due to altered chemical or physical properties of the damaged tissue.

The results of this study showed that the alcohol-loaded hydrogel was effective at chemically ablating a human uterus to a depth of about 2-5 mm along the entire uterine surface. The alcohol-loaded hydrogel was observed to adhere strongly to ablated uterine tissue, and the hydrogel ablation efficacy seems to be related to installation volume. This suggests exceeding unstretched uterine fill volumes for enhanced efficacy of ablation.

### Further Inventive Concepts

A1. A hydrogel comprising an insoluble crosslinked hydrophilic polymer with linear hydrophilic polymer arms and biodegradable linkages between one or more of the hydrophilic polymer arms, wherein the hydrogel is imbibed/infused with a fluid comprising a tissue ablation composition at a concentration suitable to ablate tissue in contact with the hydrogel during a time period prior to disintegration of the hydrogel.
A2. The hydrogel of inventive concept A1 further comprising biodegradable microparticles entrapped in the hydrogel, wherein the microparticles contain the tissue ablation composition.
A3. The hydrogel of inventive concept A2 wherein the biodegradable microparticles comprise a poly(hydroxy acid), a poly(lactone), poly(glycolic acid), poly(lactic acid), poly(lactic acid)-co-poly(glycolic acid) (PLGA), or mixtures thereof.
A4. The hydrogel of inventive concept A1 wherein the linear hydrophilic polymer arms comprise polyethylene glycol, polyvinyl alcohol, polyoxazoline, polyvinylpyrrolidone, polysaccharides, poloxamers, copolymers thereof, or mixtures thereof.
A5. The hydrogel of inventive concept A1 wherein the biodegradable linkages are chemically hydrolysable under physiological conditions.
A6. The hydrogel of inventive concept A1 wherein the biodegradable linkages are enzymatically degradable.
A7. The hydrogel of inventive concept A1 wherein the biodegradable linkages comprise an ester, a carbonate, or an amide.
A8. The hydrogel of inventive concept A1 wherein the tissue ablation composition comprises an alcohol or an acetic acid.
A9. The hydrogel of inventive concept A1 wherein the tissue ablation composition comprises acetic acid, trichloroacetic acid, or a combination thereof.
A10. The hydrogel of inventive concept A1 wherein the tissue ablation composition comprises ethanol, isopropyl alcohol, or a combination thereof.
A11. The hydrogel of inventive concept A1 wherein the fluid has a concentration of the tissue ablation composition of about 50 vol % to about 95 vol %.
A12. The hydrogel of inventive concept A1 wherein the time period is from about 2 hours to about 7 days.
A13. The hydrogel of inventive concept A1 wherein the hydrogel fully biodegrades in physiological fluid in from about 0.5 days to about 45 days.
A14. The hydrogel of inventive concept A1 wherein the hydrogel fully biodegrades in physiological fluid in from about 3 days to about 29 days.
A15. The hydrogel of inventive concept A1 wherein the hydrogel fully biodegrades in physiological fluid in from about 24 to 72 hours.
A16. The hydrogel of inventive concept A1 wherein the concentration is suitable to ablate tissue comprising uterine tissue and/or fallopian tube tissue.
A17. The hydrogel of inventive concept A2 wherein the microparticles comprise polylactic acid, polyglycolic acid, a copolymer thereof or a mixture thereof.
A18. The hydrogel of inventive concept A2 wherein the microparticles comprise polylactic acid, polyglyclic acid, a copolymer thereof or a mixture thereof, and the tissue ablation composition comprises acetic acid, trichloroacetic acid, or a combination thereof.
A19. The hydrogel of inventive concept A1 wherein the hydrogel has a Young's modulus from about 5 kPa to about 300 kPa.
A20. The hydrogel of inventive concept A1 wherein the hydrogel swells in contact with physiological fluids from an initially gelled state from about −25 wt % to about 50 wt %, which is evaluated through contact with buffered saline after 24 hours.
A21. The hydrogel of inventive concept A1 wherein hydrogel comprises cores of the multi-armed polyethyelene glycol moieties having from 4 to 8 arms and a molecular weight between crosslinks from about 400 Da to about 10,000 Da.
B. A method for forming a hydrogel system for tissue ablation comprising:
combining engorged microparticles with a solution of dissolved hydrogel precursors to form a hydrogel precursor solution, wherein the engorged microparticles are formed by absorbing a tissue ablation composition into polymer microparticles;
assembling an applicator with a reservoir of hydrogel precursor solution and a reservoir of activator solution configured for delivery of a mixture of hydrogel precursor solution and activator solution into a patient.
   B1. The method of inventive concept B wherein the polymer microparticles comprise a poly(hydroxy acid), a poly(lactone), poly(glycolic acid), poly(lactic acid), a poly(lactic acid)-co-poly(glycolic acid) (PLGA), a polycarbonate, or mixtures thereof and the tissue ablation composition comprises an alcohol or an acetic acid.
   B2. The method of inventive concept B wherein the tissue ablation composition comprises acetic acid, trichloroacetic acid, methanol, ethanol, isopropyl alcohol, or a combination thereof.
   B4. The method of inventive concept B wherein the tissue ablation composition comprises a fluid and wherein absorbing comprises soaking the polymer microparticles in the fluid.
   B5. The method of inventive concept B4 wherein the absorbing is performed for at least about 10 minutes.
   B6. The method of inventive concept B wherein the absorbing is performed using electrospraying.
   B7. The method of inventive concept B further comprising forming the polymer microparticles by milling a polymer to form particles of desired size.
   B8. The method of inventive concept B wherein the absorbing is performed during forming the polymer microparticles.
   B9. The method of inventive concept B8 wherein the absorbing comprises forming the polymer microparticles comprises polymerizing and/or crosslinking a precursor composition in the presence of the tissue ablation composition to form the engorged microparticles.
   B10. The method of inventive concept B8 wherein forming the polymer microparticles comprises mixing a polymer which is insoluble in water with the tissue ablation composition and adding water to form the engorged microparticles by phase separation.
   B11. The method of inventive concept B wherein the solution of dissolved hydrogel precursors comprises a polyfunctional hydrophilic precursor macromolecular composition.
   B12. The method of inventive concept B11 wherein the polyfunctional hydrophilic precursor macromolecular composition comprises polyethylene glycol, polyvinyl alcohol, polyoxazoline, polyvinylpyrrolidone, polysaccharides, poloxamers, copolymers thereof, or mixtures thereof.
   B13. The method of inventive concept B11 wherein the polyfunctional hydrophilic precursor macromolecular composition comprises a first precursor comprising a plurality of electrophilic functional groups and a first hydrophilic core and a second precursor comprising a plurality of nucleophilic functional groups and a second hydrophilic core, and wherein the activator solution comprises an alkaline solution.
   B14. The method of inventive concept B13 wherein the alkaline solution comprises a buffer solution having a pH greater than 8.2 and a sufficient buffer capacity to raise the pH of the mixture of hydrogel precursor solution and activator solution above a pH of 8.
   B15. The method of inventive concept B11 wherein the polyfunctional hydrophilic precursor macromolecular composition comprises free radical polymerizable functional groups, and wherein the hydrogel precursor solution comprises an initiator or a co-initiator, and wherein the activator solution comprises an initiator if the hydrogel precursor solution comprises a co-initiator or a co-initiator if the hydrogel precursor solution comprises an initiator.
   B16. The method of inventive concept B wherein the applicator comprises a first syringe containing the reservoir of hydrogel precursor solution and a second syringe containing the reservoir of activator solution, wherein the first syringe and the second syringe are connected to a catheter.
   B17. The method of inventive concept B16 wherein the catheter is no more than 3 mm in diameter.
   C1. A method for ablating tissue in a localized domain within a patient, the method comprising: delivering a hydrogel formation solution comprising a dissolved blend of a polyfunctional hydrophilic hydrogel precursor, an activator, a solvent and a tissue ablation composition into a target localized domain in a patient, wherein a hydrogel forms in contact with a target tissue for ablation by the tissue ablation composition.
   C2. The method of inventive concept C1 wherein the target tissue comprises a fallopian tube lumen.
   C3. The method of inventive concept C2 wherein the method provides permanent female sterilization.
   C4. The method of inventive concept C2 further comprising performing a tubal patency test after a selected period of time.
   C5. The method of inventive concept C1 wherein the target tissue comprises a uterine lining, a uterine fibroid, a cervical lesion, a gential wart, or a combination thereof.
   C6. The method of inventive concept C1 wherein the hydrogel swells to contact conformally with the target tissue.
   C6′. The method of inventive concept C6 wherein the target tissue is the interior of a uterus and wherein the hydrogel swells to conformally fill the uterus whether or not anatomical anomalies are present.
   C6″. The method of inventive concept C6 wherein the ablation composition dissipates while the hydrogel maintains contact with the target tissue.
   C7. The method of inventive concept C1 wherein delivering comprises directing the dissolved blend into a transcervical positioned catheter to deliver the dissolved blend into the target localized domain.
   C8. The method of inventive concept C7 wherein the cervix is not dilated.
   C9. The method of inventive concept C1 wherein the polyfunctional hydrophilic hydrogel precursor comprises polyethylene glycol, polyvinyl alcohol, polyoxazoline, polyvinylpyrrolidone, polysaccharides, poloxamers, copolymers thereof, or mixtures thereof.
   C10. The method of inventive concept C1 wherein the tissue ablation composition comprises an alcohol or an acetic acid.
   C11. The method of inventive concept C1 wherein the solvent comprises water for injection (WFI) or saline.
   C12. The method of inventive concept C1 wherein the hydrogel formation solution comprises a macromolecular composition comprising free radical polymerizable functional groups, and the activator comprises an initiator and a co-initiator.
   C13. The method of inventive concept C12 wherein the initiator comprises a peroxide group, an alkyl hydrogen peroxide group, or a persulfate, the co-initiator comprises a metal reductant, and the tissue ablation composition comprises an acetic acid.
   C14. The method of inventive concept C12 wherein a first syringe comprises the polyfunctional hydrophilic hydrogel precursor, the tissue ablation composition, and the initiator or the co-initiator and a second syringe comprises the initiator if the first syringe comprises the co-initiator or the co-initiator if the first syringe comprises the initiator, wherein the first syringe and the second syringe are connected by a plunger cap, and wherein delivering comprises directing the dissolved blend into a positioned catheter by pressing the plunger cap.
   C15. The method of inventive concept C1 wherein the hydrogel formation solution comprises a macromolecular composition comprising a first precursor comprising a plurality of electrophilic functional groups and a first hydrophilic core and a second precursor comprising a plurality of nucleophilic functional groups and a second hydrophilic core, and wherein the activator comprises an alkaline solution.
   C16. The method of inventive concept C15 wherein a first syringe comprises the polyfunctional hydrophilic hydrogel precursor and the tissue ablation composition and a second syringe comprises a buffer solution having a pH greater than 8.2, wherein the first syringe and the second syringe are connected by a plunger cap, and wherein delivering comprises directing the dissolved blend into a positioned catheter by pressing the plunger cap, wherein the dissolved blend has a pH above 8.
   C17. The method of inventive concept C15 wherein the hydrogel formation solution further comprises polymer microparticles, wherein the polymer microparticles sequester the tissue ablation composition.
   C18. The method of inventive concept C1 wherein the hydrogel formation solution further comprises a visualization agent.
   C19. The method of inventive concept C18 further comprising removing the hydrogel formation solution and/or the hydrogel from a tissue that is not the target tissue.
   C20. The method of inventive concept C18 further comprising visualizing ablation of the target tissue using an imaging device.
   C21. The method of inventive concept C1 wherein the hydrogel formation solution has a gel time of no more than about 20 seconds.
   C22. The method of inventive concept C1 wherein the target tissue comprises a tumor or precancerous lesion.
   D1. A kit for forming tissue ablative hydrogels, the kit comprising:
a powder of a dry polyfunctional hydrophilic polymer with a plurality of crosslinkable functional groups;
a liquid comprising a tissue ablation composition, wherein the powder is soluble in the liquid; and
a container comprising an activator,
   wherein upon dissolving the powder in the liquid to form a hydrogel precursor solution and mixing the hydrogel precursor solution with the activator, a hydrogel formation solution is formed that gels into a biodegradable hydrogel comprising absorbed tissue ablation composition.
   D2. The kit of inventive concept D1 further comprising:
polymer microparticles insoluble in water, wherein the polymer microparticles soaked in the tissue ablation composition imbibe the tissue ablation composition to form engorged microparticles and wherein the engorged microparticles can be dispersed into the hydrogel formation solution for the formation of a biodegradable hydrogel loaded with engorged microparticles.
   D3. The kit of inventive concept D1 wherein the activator comprises an alkaline composition.
   D4. The kit of inventive concept D1 wherein the activator comprises a peroxide or a peroxide reducing agent and wherein the hydrogel precursor solution further comprises a co-activator that is a peroxide reducing agent if the activator is a peroxide or a peroxide if the activator is a peroxide reducing agent.
   D5. The kit of inventive concept D1 wherein the dry polyfunctional hydrophilic polymer comprises polyethylene glycol, polyvinyl alcohol, polyoxazoline, polyvinylpyrrolidone, polysaccharides, poloxamers, copolymers thereof, or mixtures thereof.
   D6. The kit of inventive concept D1 wherein combining the powder and the liquid forms the hydrogel-based system of any one of claims A1-A34 wherein the solution formed from the powder and the liquid is the hydrogel precursor solution.
   E1. A method for forming a hydrogel precursor system for delivery of a tissue ablating hydrogel, the method comprising:
mixing a) a powder of a dry polyfunctional hydrophilic polymer with a plurality of crosslinkable functional groups and b) a liquid comprising a tissue ablation composition to dissolve the powder and form a hydrogel precursor solution; and
providing the hydrogel precursor solution and an activator solution in a suitable applicator designed to mix the hydrogel precursor solution and the activator solution while delivering a blend of the hydrogel precursor solution and the activator solution to a target location within a patient.
   E2. The method of inventive concept E1 further comprising:
mixing a) and b) with c) polymer microparticles insoluble in water, wherein the polymer microparticles associated with the tissue ablation composition imbibe the tissue ablation composition within engorged microparticles to form the hydrogel precursor solution with dispersed engorged microparticles.
   E3. The method of inventive concept E1 wherein the activator solution comprises an alkaline composition.
   E4. The method of inventive concept E1 wherein the activator solution comprises a peroxide or a peroxide reducing agent and wherein the hydrogel precursor solution further comprises a co-activator that is a peroxide reducing agent if the activator is a peroxide or a peroxide if the activator is a peroxide reducing agent.
   E5. The method of inventive concept E1 wherein the dry polyfunctional hydrophilic polymer comprises branched macromolecules having arms comprising polyethylene glycol, polyvinyl alcohol, polyoxazoline, polyvinylpyrrolidone, polysaccharides, poloxamers, copolymers thereof, or mixtures thereof.
   E6. The method of inventive concept E5 wherein the dry multifunctional hydrophilic polymer comprises a plurality of electrophilic functional groups and wherein mixing comprises mixing with c) a hydrogel precursor comprising a plurality of nucleophilic functional groups.
   E7. The method of inventive concept E5 wherein the dry multifunctional hydrophilic polymer comprises a plurality of crosslinkable vinyl groups.
   E8. The method of inventive concept E1 wherein the suitable applicator comprises a first syringe comprising the hydrogel precursor solution and a second syringe comprising the activator solution, wherein the first syringe and the second syringe are connected by a plunger cap, and wherein delivering comprises directing the blend into a positioned catheter by pressing the plunger cap.
   F1. A method for performing ablation of a uterine lining of a patient, the method comprising:
delivering a hydrogel formation solution through a catheter into a uterine cavity of the patient with an appropriate volume to fill the cavity without significant flow into the fallopian tubes and with restrictions to inhibit flow into the cervix, wherein the hydrogel formation solution gels in no more than 20 seconds into a hydrogel engorged with tissue ablation composition at a concentration sufficient to ablate tissue in contact with the hydrogel.
   F2. The method of inventive concept F1 wherein the hydrogel formation solution comprises a polyfunctional hydrophilic polymer with a plurality of crosslinkable functional groups, an activator, and a tissue ablation composition.
   F3. The method of inventive concept F2 wherein the crosslinkable functional groups comprise an acrylate group and/or a methacrylate group and the activator comprises a peroxide and a peroxide reductant.
   F4. The method of inventive concept F2 wherein the hydrogel formation solution comprises a first polyfunctional hydrophilic polymer with a plurality of electrophilic functional groups and a second polyfunctional hydrophilic polymer with a plurality of nucleophilic functional groups, and the activator comprises an alkaline composition.
   F5. The method of inventive concept F4 wherein the electrophilic functional groups comprise succinimidyl succinate, succinimidyl succinamide, succinimidyl glutarate, succinimidyl glutaramide, succinimidyl adipate, succinimidyl azelate, or a combination thereof.
   F6. The method of inventive concept F4 wherein the nucleophilic functional groups comprise a protonated amine.
   F7. The method of inventive concept F1 wherein the hydrogel formation solution comprises a first polyethylene glycol having a molecular weight from about 5K to about 40K and from 3 to 10 arms, wherein the arms are terminated by succinimidyl glutarate (SG) functional groups or succinimidyl succinate (SS) functional groups, and a second polyethylene glycol having a molecular weight from about 5K to about 40K and from 3 to 10 arms, wherein the arms are terminated by an HCl-salted amine.
   F8. The method of inventive concept F1 wherein the appropriate volume comprises a volume that is from about 11% to about 200% of the volume of the undistended uterus.
   F9. The method of inventive concept F1 wherein delivering is performed using an applicator comprising a catheter configured for transcervical intrauterine placement with a cap element on the catheter to allow for stopping egress from the cervix.
   F10. The method of inventive concept F1 wherein the tissue ablation composition comprises an aqueous solution having from about 50 vol % to about 95 vol % alcohol or acetic acid.
   F11. The method of inventive concept F1 wherein the hydrogel swells from about −25 wt % to about 200 wt % after formation.
   F12. The method of inventive concept F1 wherein the hydrogel conformably fills the uterus.
   F13. The method of inventive concept F1 wherein the hydrogel comprises a covalently crosslinked moieties comprising polyethylene glycol, polyvinyl alcohol, polyoxazoline, polyvinylpyrrolidone, polysaccharides, poloxamers, copolymers thereof, or mixtures thereof.
   F14. The method of inventive concept F1 wherein the tissue ablation composition comprises ethanol.
   F15. The method of inventive concept F1 wherein the hydrogel comprises a covalently crosslinked polymer with degradable linkages, wherein the biodegradable linkages comprise an ester, a carbonate, or an amide.
   F17. The method of inventive concept F1 wherein the hydrogel fully biodegrades or is expunged from the uterus in about 3 days to about 29 days.
   F18. The method of inventive concept F1 wherein the hydrogel has a persistence that prevents uterine adhesions.
   F19. The method of inventive concept F1 wherein the untreated uterus comprises fibroids.
   F20. The method of inventive concept F19 wherein the delivery of the hydrogel formation solution results in the ablation of the fibroids.
   F21. The method of inventive concept F1 wherein the alcohol is substantially displaced from the hydrogel prior to the expulsion of the hydrogel from the uterus due to hydrolytic degradation and/or expulsion from the uterine cavity.

The embodiments above are intended to be illustrative and not limiting. Additional embodiments are within the claims. In addition, although the present invention has been described with reference to particular embodiments, those skilled in the art will recognize that changes can be made in form and detail without departing from the spirit and scope of the invention. Any incorporation by reference of documents above is limited such that no subject matter is incorporated that is contrary to the explicit disclosure herein. To the extent that specific structures, compositions and/or processes are described herein with components, elements, ingredients or other partitions, it is to be understood that the disclosure herein covers the specific embodiments, embodiments comprising the specific components, elements, ingredients, other partitions or combinations thereof as well as embodiments consisting essentially of such specific components, ingredients or other partitions or combinations thereof that can include additional features that do not change the fundamental nature of the subject matter, as suggested in the discussion, unless otherwise specifically indicated.

What is claimed is:

1. A hydrogel-based system for chemical tissue ablation comprising:
a hydrogel precursor solution comprising a polyfunctional hydrophilic precursor macromolecular composition in a solution comprising a tissue ablation composition; and
an activator solution,
wherein mixing the hydrogel precursor solution and the activator solution results in the crosslinking of the polyfunctional hydrophilic polymer to form a fluid imbibed/infused hydrogel with the fluid comprising the tissue ablation composition at a concentration suitable to ablate tissue in contact with the fluid imbibed/infused hydrogel.

2. The hydrogel-based system of claim 1 wherein the tissue ablation composition comprises an alcohol or an acetic acid.

3. The hydrogel-based system of claim 1 wherein the tissue ablation composition comprises acetic acid, trichloroacetic acid, methanol, ethanol, isopropyl alcohol, or a combination thereof.

4. The hydrogel-based system of claim 1 wherein the hydrogel precursor solution comprises biodegradable microparticles containing the tissue ablation composition.

5. The hydrogel-based system of claim 4 wherein the biodegradable microparticles comprise a poly(hydroxy acid), a poly(lactone), poly(glycolic acid), poly(lactic acid), a poly(lactic acid)-co-poly(glycolic acid) (PLGA), a polycarbonate, or mixtures thereof.

6. The hydrogel-based system of claim 1 wherein the polyfunctional hydrophilic precursor macromolecular composition comprises a first precursor comprising a plurality of electrophilic functional groups and a first hydrophilic core and a second precursor comprising a plurality of nucleophilic functional groups and a second hydrophilic core.

7. The hydrogel-based system of claim 6 wherein the ratio of electrophilic functional groups to nucleophilic functional groups is from 0.8:1.0 to 1.0:0.8.

8. The hydrogel-based system of claim 6 wherein the first hydrophilic core and/or the second hydrophilic core comprise polyethylene glycol, polyvinyl alcohol, polyoxazoline, polyvinylpyrrolidone, polysaccharides, poloxamers, copolymers thereof, or mixtures thereof.

9. The hydrogel-based system of claim 6 wherein the first hydrophilic core has a plurality of arms having electrophilic functional groups, the second hydrophilic core has a plurality of arms having nucleophilic functional groups, or a combination thereof.

10. The hydrogel-based system of claim 9 wherein the plurality of arms is from 3 to 10.

11. The hydrogel-based system of claim 9 wherein the nucleophilic functional groups comprise protonated amine groups.

12. The hydrogel-based system of claim 6 wherein the first precursor and the second precursor independently have a molecular weight of about 2K Da to about 50K Da and from 3 to 8 arms.

13. The hydrogel-based system of claim 6 wherein the electrophilic functional groups comprise a reactive ester.

14. The hydrogel-based system of claim 6 wherein the electrophilic functional groups comprise succinimidyl succinate, succinimidyl succinamide, succinimidyl glutarate, succinimidyl glutaramide, succinimidyl adipate, succinimidyl azelate, or a combination thereof.

15. The hydrogel-based system of claim 6 wherein the electrophilic functional groups have a degradable ester linkage to the first hydrophilic core.

16. The hydrogel-based system of claim 6 wherein the hydrogel precursor solution has a pH from about 4.0 to about 6.2.

17. The hydrogel-based system of claim 6 wherein the activator solution comprises a buffer solution having a pH greater than 8.2 and a sufficient buffer capacity to raise the pH of a mixture of the hydrogel precursor solution and the activator solution above a pH of 8.

18. The hydrogel-based system of claim 6 wherein the hydrogel precursor solution comprises biodegradable microparticles containing the tissue ablation composition.

19. The hydrogel-based system of claim 18 wherein the tissue ablation composition comprises ethanol.

20. The hydrogel-based system of claim 1 wherein the polyfunctional hydrophilic precursor macromolecular composition comprises free radical polymerizable functional groups, wherein the hydrogel precursor solution comprises an initiator or a co-initiator, and wherein the activator solution comprises an initiator if the hydrogel precursor solution comprises a co-initiator or a co-initiator if the hydrogel precursor solution comprises an initiator.

21. The hydrogel-based system of claim 20 wherein the free radical polymerizable functional groups comprise an acrylate functional group, a methacrylate functional group, and/or derivatives thereof.

22. The hydrogel-based system of claim 20 wherein the polyfunctional hydrophilic precursor macromolecular composition comprises polyethylene glycol diacrylate.

23. The hydrogel-based system of claim 20 wherein the initiator comprises a peroxide group, an organic hydroperoxide group, or a persulfate, and the co-initiator comprises a metal reductant.

24. The hydrogel-based system of claim 20 wherein the co-initiator comprises Fe2+, Cr2+, V2+, Ti3+, Co2+, or Cu+, or a combination thereof.

25. The hydrogel-based system of claim 20 wherein the initiator comprises dicumyl peroxide and the co-initiator comprises Fe2+.

26. The hydrogel-based system of claim 20 wherein the tissue ablation composition comprises an acetic acid.

27. The hydrogel-based system of claim 1 wherein the hydrogel precursor solution comprising a solvent with from about 80% to about 100% by volume of the tissue ablation composition.

28. The hydrogel-based system of claim 1 wherein the hydrogel precursor solution and/or the activator solution are a solution comprising a tissue ablation composition and wherein the solution comprising a tissue ablation composition comprises from about 0% to about 30% by volume of water.

29. The hydrogel-based system of claim 1 wherein the hydrogel precursor solution has a concentration of the tissue ablation composition from about 50 vol % to about 95 vol %.

30. The hydrogel-based system of claim 1 wherein the hydrogel precursor solution has a solids content from about 3 wt % to about 25 wt %.

31. The hydrogel-based system of claim 1 further comprising a visualization agent.

32. The hydrogel-based system of claim 31 wherein the visualization agent is biocompatible and comprises a coloring agent, a fluorescent molecule, a contrast agent, or a combination thereof.

33. The hydrogel-based system of claim 1 wherein the hydrogel precursor solution further comprises a coloring agent.

34. The hydrogel-based system of claim 1 wherein the hydrogel precursor solution further comprises a therapeutic agent.

35. The hydrogel-based system of claim 1 wherein the hydrogel precursor solution and the activator solution are formed in a volume ratio from about 1:1 to about 20:1.

36. The hydrogel-based system of claim 1 wherein a formation solution for chemical tissue ablation comprises a blend of the hydrogel precursor solution and the activator solution.

37. The hydrogel-based system of claim 36 wherein the blend comprises the hydrogel precursor solution and the activator solution in a volume ratio from about 1:1 to about 20:1.
